(19) United States
(12) Patent Application Publication
(10) Pub. No.: US 2023/0203513 A1
(43) Pub. Date: Jun. 29, 2023

(54) CUCUMBER PLANT HABIT

(71) Applicant: BETTERSEEDS LTD, Givat Chen (IL)

(72) Inventors: Ido MARGALIT, Gan Yavne (IL); Tal SHERMAN, Rehovot (IL)

(73) Assignee: BETTERSEEDS LTD, Givat Chen (IL)

(21) Appl. No.: 17/995,929

(22) PCT Filed: Apr. 12, 2021
(86) PCT No.: PCT/IL2021/050415

§ 371 Date: Oct. 11, 2022

Related U.S. Application Data
(60) Provisional application No. 63/008,752, filed on Apr. 12, 2020.

(51) Int. Cl. C12N 15/82; C12N 15/11

(52) U.S. Cl. CPC C12N 15/8213; C12N 15/8261; C12N 15/11; C12N 2310/20

(57) ABSTRACT

Provided relates to conferring desirable agronomic traits in Cucumber plants. Further, provided is a modified Cucumber plant exhibiting at least one improved domestication trait. The modified Cucumber plant includes at least one genetic modification conferring reduced expression of at least one Cucumber SELF PRUNING (SP) (CuSP) gene. Also, further provided are methods for producing the aforementioned modified Cucumber plant and uses thereof.

### FIELD OF THE INVENTION

The present disclosure relates to conferring desirable agronomic traits in Cucumber plants. More particularly, the current invention pertains to producing Cucumber plants with improved yield traits by manipulating genes controlling day-length sensitivity and plant architecture.

### BACKGROUND OF THE INVENTION

One of the most important determinants of crop productivity is plant architecture. For many crops, artificial selection for modified shoot architectures provided critical steps towards improving yield, followed by innovations enabling large-scale field production. A prominent example is tomato, in which the discovery of a mutation in the antiflorigen-encoding self-pruning gene (sp), led to determinate plants that provided a burst of flowering and synchronized fruit ripening, permitting mechanical harvesting.

The publication of Li et al (2018) teaches the assembly of a set of six gRNAs to edit four genes (SlCLV3, SlWUS, SP and SP5G) into one construct. The construct was transformed into four S. pimpinellifolium accessions, all of which are resistant to bacterial spot disease, and two of which are salt tolerant. Small indels and large insertions have been identified in the targeted regulatory regions of SlCLV3 and SlWUS in T0 and their T1 mutant plants. It was reported in this publication that although SP and SP5G are crucial for improving the harvest index, the limited allelic variation has hampered efforts to optimize this trait. It was further reported that locule number was not increased in T0 and T1 plants with large insertions and inversions in the targeted SlCLV3 promoter region. One explanation for this finding is that the targeted region of the SlCLV3 promoter may not be essential for regulating SlCLV3 transcription. Alternatively, it was suggested that disruption of regions flanking the CArG transcription-repressor element downstream of SlWUS may have decreased its transcription and counteracted the effects of mutation of SlCLV3, owing to a negative feedback loop of small-peptide-encoding gene CLV3 (CLAVATA3) CLV3 and the homeobox-encoding gene WUS (WUSCHEL), in controlling stem cell proliferation.

The publication of Zsögön et al (2018) discloses a devised CRISPR-Cas9 genome engineering strategy to combine agronomically desirable traits with useful traits presented in Solanum pimpinellifolium wild lines. The four edited genes were SELF-PRUNING (SP), OVATE (O), FRUIT WEIGHT 2.2 (FW2.2) and LYCOPENE BETA CYCLASE (CycB).

Lemmon et al (2018) describes the usage of CRISPR-Cas9 to mutate orthologues of tomato domestication and improvement genes that control plant architecture, flower production and fruit size in the orphan Solanaceae crop ‘groundcherry’ (Physalis pruinosa).

In view of the above there is still a long felt and unmet need to manipulate Cucumber plant architecture and flower production in a rapid and efficient way to improve yield and reduce production costs.

### SUMMARY OF THE INVENTION

It is therefore one object of the present invention to disclose a modified Cucumber plant exhibiting at least one improved domestication trait, wherein said modified plant comprises at least one genetic modification conferring reduced expression of at least one Cucumber SELF PRUNING (SP) (CuSP) gene.

It is a further object of the present invention to disclose the modified Cucumber plant as defined in any of the above, wherein said CuSP gene is selected from the group consisting of CuSP-1 having a genomic nucleotide sequence as set forth in SEQ ID NO:1 or a functional variant or homologue thereof, CuSP-2 having a genomic nucleotide sequence as set forth in SEQ ID NO:89 or a functional variant or homologue thereof, CuSP-3 having a genomic nucleotide sequence as set forth in SEQ ID NO: 167 or a functional variant or homologue thereof and any combination thereof.

It is a further object of the present invention to disclose the modified Cucumber plant as defined in any of the above, wherein said functional variant or homologue has at least 75% sequence identity to said CuSP nucleotide sequence.

It is a further object of the present invention to disclose the modified Cucumber plant as defined in any of the above, wherein said modified cucumber plant exhibits at least one improved domestication trait as compared to a corresponding Cucumber plant lacking said genetic modification.

It is a further object of the present invention to disclose the modified Cucumber plant as defined in any of the above, wherein said genetic modification is introduced using mutagenesis, small interfering RNA (siRNA), microRNA (miRNA), artificial miRNA (amiRNA), DNA introgression, endonucleases or any combination thereof.

It is a further object of the present invention to disclose the modified Cucumber plant as defined in any of the above, wherein said modified cucumber plant comprises at least one genetic modification introduced in said at least one CuSP gene using targeted genome modification.

It is a further object of the present invention to disclose the modified Cucumber plant as defined in any of the above, wherein said genetic modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further object of the present invention to disclose the modified Cucumber plant as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas010, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

It is a further object of the present invention to disclose the modified Cucumber plant as defined in any of the above, wherein the genetically modified CuSP gene is a CRISPR/Cas9—induced heritable mutated allele.

It is a further object of the present invention to disclose the modified Cucumber plant as defined in any of the above, wherein said genetic modification is a missense mutation, nonsense mutation, insertion, deletion, indel, substitution or duplication.

It is a further object of the present invention to disclose the modified Cucumber plant as defined in any of the above, wherein the insertion or the deletion produces a gene comprising a frameshift.

It is a further object of the present invention to disclose the modified Cucumber plant as defined in any of the above, wherein said plant is homozygous for said at least one genetically modified CuSP gene.

It is a further object of the present invention to disclose the modified Cucumber plant as defined in any of the above, wherein said genetic modification is in the coding region of said gene, a mutation in the regulatory region of said gene, or an epigenetic factor.

It is a further object of the present invention to disclose the modified Cucumber plant as defined in any of the above, wherein said genetic modification is a silencing mutation, a knockdown mutation, a knockout mutation, a loss of function mutation or any combination thereof.

It is a further object of the present invention to disclose the modified Cucumber plant as defined in any of the above, wherein said genetic modification is generated in planta.

It is a further object of the present invention to disclose the modified Cucumber plant as defined in any of the above, wherein said genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO:4-88, 92-166, 170-255 and any combination thereof.

It is a further object of the present invention to disclose the modified Cucumber plant as defined in any of the above, wherein said genetic modification in said CuSP-1 is generated in planta via introduction of a construct comprising (a) Cas DNA and gRNA sequence selected from the group consisting of SEQ ID NO:4-88 and any combination thereof, or (b) a ribonucleoprotein (RNP) complex comprising Cas protein and gRNA sequence selected from the group consisting of SEQ ID NO:4-88 and any combination thereof.

It is a further object of the present invention to disclose the modified Cucumber plant as defined in any of the above, wherein said mutation in said CuSP-2 is generated in planta via introduction of a construct comprising (a) Cas DNA and gRNA sequence selected from the group consisting of SEQ ID NO:92-166 and any combination thereof, or (b) a ribonucleoprotein (RNP) complex comprising Cas protein and gRNA sequence selected from the group consisting of SEQ ID NO:92-166 and any combination thereof.

It is a further object of the present invention to disclose the modified Cucumber plant as defined in any of the above, wherein said mutation in said CuSP-3 is generated in planta via introduction of a construct comprising (a) Cas DNA and gRNA sequence selected from the group consisting of SEQ ID NO:170-SEQ ID NO:255 and any combination thereof, or (b) a ribonucleoprotein (RNP) complex comprising Cas protein and gRNA sequence selected from the group consisting of SEQ ID NO:170-255 and any combination thereof.

It is a further object of the present invention to disclose the modified Cucumber plant as defined in any of the above, wherein said gRNA sequence comprises a 3′ NGG Protospacer Adjacent Motif (PAM).

It is a further object of the present invention to disclose the modified Cucumber plant as defined in any of the above, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is a further object of the present invention to disclose the modified Cucumber plant as defined in any of the above, wherein said plant has decreased expression levels of at least one of said CuSP genes.

It is a further object of the present invention to disclose the modified Cucumber plant as defined in any of the above, wherein the sequence of said expressed CuSP gene is selected from the group consisting of: SEQ ID NO:2, SEQ ID NO:3, SEQ ID NO:90, SEQ ID NO:91, SEQ ID NO:168 and SEQ ID NO: 169 or a functional variant or homologue thereof.

It is a further object of the present invention to disclose the modified Cucumber plant as defined in any of the above, wherein said plant is semi-determinant.

It is a further object of the present invention to disclose the modified Cucumber plant as defined in any of the above, wherein said plant has determinant growth habit.

It is a further object of the present invention to disclose the modified Cucumber plant as defined in any of the above, wherein said plant flowers earlier than a corresponding Cucumber plant lacking said genetic modification.

It is a further object of the present invention to disclose the modified Cucumber plant as defined in any of the above, wherein said plant exhibits improved earliness as compared to a corresponding Cucumber plant lacking said genetic modification.

It is a further object of the present invention to disclose the modified Cucumber plant as defined in any of the above, wherein said plant exhibits suppressed sympodial shoot termination as compared to a corresponding Cucumber plant lacking said genetic modification.

It is a further object of the present invention to disclose the modified Cucumber plant as defined in any of the above, wherein said plant exhibits similar sympodial shoot termination as compared to a corresponding Cucumber plant lacking said genetic modification.

It is a further object of the present invention to disclose the modified Cucumber plant as defined in any of the above, wherein said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is a further object of the present invention to disclose a Cucumber plant, plant part, plant fruit or plant cell as defined in any of the above, wherein said plant does not comprise a transgene.

It is a further object of the present invention to disclose a plant part, plant cell, plant fruit or plant seed of a modified cucumber plant as defined in any of the above, wherein said plant part, plant cell, plant fruit or plant seed comprises at least one genetic modification conferring reduced expression of at least one Cucumber SELF PRUNING (SP) (CuSP) gene.

It is a further object of the present invention to disclose a tissue culture of regenerable cells, protoplasts or callus obtained from the modified Cucumber plant as defined in any of the above.

It is a further object of the present invention to disclose the modified Cucumber plant as defined in any of the above, wherein said plant genotype is obtainable by deposit under accession number with NCIMB Aberdeen AB21 9YA, Scotland, UK or with ATCC.

It is a further object of the present invention to disclose a method for producing a modified Cucumber plant exhibiting at least one improved domestication trait, wherein said method comprises steps of genetically modifying at least one Cucumber SELF PRUNING (SP) (CuSP) gene.

It is a further object of the present invention to disclose the method as defined in any of the above, comprises steps of producing the modified Cucumber plant using targeted genome modification, by genetically introducing a loss of function mutation in said at least one Cucumber SELF PRUNING (SP) (CuSP) gene.

It is a further object of the present invention to disclose the method as defined in any of the above, wherein said genetic modification confers reduced expression of at least one Cucumber SELF PRUNING (SP) (CuSP) gene.

It is a further object of the present invention to disclose the method as defined in any of the above, wherein said modified cucumber plant exhibits at least one improved domestication trait as compared to a corresponding Cucumber plant lacking said genetic modification.

It is a further object of the present invention to disclose the method as defined in any of the above, wherein said method comprises steps of: (a) identifying at least one Cucumber SP (CuSP) gene or allele; (b) synthetizing at least one guide RNA (gRNA) comprising a nucleotide sequence complementary to said at least one identified CuSP allele; (c) transforming Cucumber plant cells with a construct comprising (a) Cas nucleotide sequence operably linked to said at least one gRNA, or (b) a ribonucleoprotein (RNP) complex comprising Cas protein and said at least one gRNA; (d) screening the genome of said transformed plant cells for induced targeted loss of function mutation in at least one of said CuSP allele or gene; (e) regenerating Cucumber plant carrying said loss of function mutation in at least one of said CuSP allele or gene; and (f) screening said regenerated plants for a Cucumber plant with improved domestication trait.

It is a further object of the present invention to disclose the method as defined in any of the above, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said CuSP allele or gene.

It is a further object of the present invention to disclose the method as defined in any of the above, wherein said CuSP Cucumber gene is selected from the group consisting of CuSP-1 having a genomic nucleotide sequence as set forth in SEQ ID NO: 1 or a functional variant or homologue thereof, CuSP-2 having a genomic nucleotide sequence as set forth in SEQ ID NO:89 or a functional variant or homologue thereof, CuSP-3 having a genomic nucleotide sequence as set forth in SEQ ID NO: 167 or a functional variant or homologue thereof and any combination thereof. It is a further object of the present invention to disclose the method as defined in any of the above, wherein said functional variant or homologue has at least 75% sequence identity to said CuSP nucleotide sequence.

It is a further object of the present invention to disclose the method as defined in any of the above, wherein said genetic modification is introduced using mutagenesis, small interfering RNA (siRNA), microRNA (miRNA), artificial miRNA (amiRNA), DNA introgression, endonucleases or any combination thereof.

It is a further object of the present invention to disclose the method as defined in any of the above, wherein said genetic modification is introduced using targeted gene editing.

It is a further object of the present invention to disclose the method as defined in any of the above, wherein said genetic modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further object of the present invention to disclose the method as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ(Cas-phi) and any combination thereof.

It is a further object of the present invention to disclose the method as defined in any of the above, wherein the mutated CuSP gene is a CRISPR/Cas9—induced heritable mutated allele or gene.

It is a further object of the present invention to disclose the method as defined in any of the above, wherein said genetic modification is a missense mutation, nonsense mutation, insertion, deletion, indel, substitution or duplication.

It is a further object of the present invention to disclose the method as defined in any of the above, wherein the insertion or the deletion produces a gene comprising a frameshift.

It is a further object of the present invention to disclose the method as defined in any of the above, wherein said modified plant is homozygous for said at least one CuSP mutated gene.

It is a further object of the present invention to disclose the method as defined in any of the above, wherein said genetic modification is in the coding region of said gene, a mutation in the regulatory region of said gene, or an epigenetic factor.

It is a further object of the present invention to disclose the method as defined in any of the above, wherein said genetic modification is a silencing mutation, a knockdown mutation, a knockout mutation, a loss of function mutation or any combination thereof.

It is a further object of the present invention to disclose the method as defined in any of the above, wherein said genetic modification is generated in planta.

It is a further object of the present invention to disclose the method as defined in any of the above, wherein said genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO: 4-88, 92-166, 170-255 and any combination thereof.

It is a further object of the present invention to disclose the method as defined in any of the above, wherein said mutation in said CuSP-1 is generated in planta via introduction of a construct comprising (a) Cas DNA and gRNA sequence selected from the group consisting of SEQ ID NO:4-88 and any combination thereof, or (b) a ribonucleoprotein (RNP) complex comprising Cas protein and gRNA sequence selected from the group consisting of SEQ ID NO:4-88 and any combination thereof.

It is a further object of the present invention to disclose the method as defined in any of the above, wherein said mutation in said CuSP-2 is generated in planta via introduction of a construct comprising (a) Cas DNA and gRNA sequence selected from the group consisting of SEQ ID NO:92-166 and any combination thereof, or (b) a ribonucleoprotein (RNP) complex comprising Cas protein and gRNA sequence selected from the group consisting of SEQ ID NO:92-166 and any combination thereof.

It is a further object of the present invention to disclose the method as defined in any of the above, wherein said mutation in said CuSP-3 is generated in planta via introduction of a construct comprising (a) Cas DNA and gRNA sequence selected from the group consisting of SEQ ID NO:170-255 and any combination thereof, or (b) a ribonucleoprotein (RNP) complex comprising Cas protein and gRNA sequence selected from the group consisting of SEQ ID NO:170-255 and any combination thereof.

It is a further object of the present invention to disclose the method as defined in any of the above, wherein said gRNA sequence comprises a 3′ NGG Protospacer Adjacent Motif (PAM).

It is a further object of the present invention to disclose the method as defined in any of the above, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is a further object of the present invention to disclose the method as defined in any of the above, wherein said modified plant has decreased expression levels of at least one of said CuSP genes.

It is a further object of the present invention to disclose the method as defined in any of the above, wherein the sequence of said expressed CuSP gene is selected from the group consisting of: SEQ ID NO:2, SEQ ID NO:3, SEQ ID NO:90, SEQ ID NO:91, SEQ ID NO:168 and SEQ ID NO:169 or a functional variant or homologue thereof.

It is a further object of the present invention to disclose the method as defined in any of the above, wherein said modified plant is semi-determinant.

It is a further object of the present invention to disclose the method as defined in any of the above, wherein said modified plant has determinant growth habit.

It is a further object of the present invention to disclose the method as defined in any of the above, wherein said modified plant flowers earlier than a corresponding Cucumber plant lacking said genetic modification.

It is a further object of the present invention to disclose the method as defined in any of the above, wherein said modified plant exhibits improved earliness as compared to a corresponding Cucumber plant lacking said genetic modification.

It is a further object of the present invention to disclose the method as defined in any of the above, wherein said modified plant exhibits suppressed sympodial shoot termination as compared to a corresponding Cucumber plant lacking said genetic modification.

It is a further object of the present invention to disclose the method as defined in any of the above, wherein said modified plant exhibits similar sympodial shoot termination as compared to a corresponding Cucumber plant lacking said genetic modification.

It is a further object of the present invention to disclose the method as defined in any of the above, wherein said modified plant exhibits suppressed or reduced day-length sensitivity as compared to a corresponding Cucumber plant lacking said genetic modification.

It is a further object of the present invention to disclose a modified Cucumber plant, plant part, plant fruit or plant cell produced by the method as defined in any of the above, wherein said plant does not comprise a transgene.

It is a further object of the present invention to disclose a plant part, plant cell, plant fruit or plant seed of a plant produced by the method as defined in any of the above.

It is a further object of the present invention to disclose a tissue culture of regenerable cells, protoplasts or callus obtained from the modified Cucumber plant produced by the method as defined in any of the above.

It is a further object of the present invention to disclose the method as defined in any of the above, wherein said plant genotype is obtainable by deposit under accession number with NCIMB Aberdeen AB21 9YA, Scotland, UK or with ATCC.

It is a further object of the present invention to disclose the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is a further object of the present invention to disclose an isolated nucleotide sequence having at least 75% sequence identity to a CuSP genomic nucleotide sequence selected from the group consisting of SEQ ID NO:1, SEQ ID NO:89 and SEQ ID NO:167.

It is a further object of the present invention to disclose the isolated nucleotide sequence having at least 75% sequence identity to a CuSP nucleotide coding sequence selected from the group consisting of SEQ ID NO:2, SEQ ID NO:90 and SEQ ID NO: 168.

It is a further object of the present invention to disclose an isolated amino acid sequence having at least 75% sequence similarity to a CuSP amino acid sequence selected from the group consisting of SEQ ID NO:3, SEQ ID NO:91 and SEQ ID NO:169.

It is a further object of the present invention to disclose an isolated nucleotide sequence having at least 75% sequence identity to a CuSP-targeted gRNA nucleotide sequence as set forth in SEQ ID NO:4-88, 92-166 and 170-255.

It is a further object of the present invention to disclose a use of a nucleotide sequence as set forth in at least one of SEQ ID NO:4-88 and any combination thereof for targeted genome modification of Cucumber SP-1 (CuSP-1) allele or gene.

It is a further object of the present invention to disclose a use of a nucleotide sequence as set forth in at least one of SEQ ID NO:92-166 and any combination thereof for targeted genome modification of Cucumber SP-2 (CuSP-2) allele or gene.

It is a further object of the present invention to disclose a use of a nucleotide sequence as set forth in at least one of SEQ ID NO: 170-255 and any combination thereof for targeted genome modification of Cucumber SP-3 (CuSP-3) allele or gene.

### BRIEF DESCRIPTION OF THE FIGURES

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIG. 1 is schematically presenting CRISPR/Cas9 mode of action as depicted by Xie and Yang (2013); and

FIG. 2 is photographically presenting regenerated transformed Cucumber tissue.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention provides a modified Cucumber plant exhibiting at least one improved domestication trait compared with wild type Cucumber, wherein said modified plant comprises at least one mutated Cucumber SELF PRUNING (SP) (CuSP) gene. The present invention further provides methods for producing the aforementioned modified Cucumber plant using genome editing or other genome modification techniques.

The solution proposed by the current invention is using genome editing such as the CRISPR/Cas system in order to create cultivated Cucumber plants with improved yield and more specifically with determinate growth habit. Breeding using genome editing allows a precise and significantly shorter breeding process in order to achieve these goals with a much higher success rate. Thus genome editing, has the potential to generate improved varieties faster and at a lower cost.

It is further noted that using genome editing is considered as non GMO by the Israeli regulator and in the US, the USDA has already classified a dozen of genome edited plant as non-regulated and non GMO (https://www.usda.gov/media/press-releases/2018/03/28/secretary-perdue-issues-usda-statement-plant-breeding-innovation).

Legal limitations and outdated breeding techniques significantly hamper the efforts of generating new and improved Cucumber varieties fit for intensive agriculture.

The present invention provides Cucumber plants with improved domestication traits such as plant architecture. The current invention discloses the generation of non-transgenic Cucumber plants with improved yield traits, using the genome editing technology, e.g., the CRISPR/Cas9 highly precise tool. The generated mutations can be introduced into elite or locally adapted Cucumber lines rapidly, with relatively minimal effort and investment.

Genome editing is an efficient and useful tool for increasing crop productivity, and there is particular interest in advancing manipulation of domestication genes in Cucumber wild species, which often have undesirable characteristics.

Genome-editing technologies, such as the Clustered Regularly Interspaced Short Palindromic Repeats (CRISPR)-CRISPR-associated protein-9 nuclease (Cas9) (CRISPR-Cas9) provide opportunities to address these deficiencies, with the aims of increasing quality and yield, improve adaptation and expand geographical ranges of cultivation.

To that end, guide RNAs (gRNAs) were designed for each of the target genes identified in Cucumber to induce mutations in SP through genome editing.

According to one embodiment, the present invention provides a modified Cucumber plant exhibiting at least one improved domestication trait, wherein said modified plant comprises at least one genetic modification conferring reduced expression of at least one Cucumber SELF PRUNING (SP) (CuSP) gene.

According to a further embodiment of the present invention, the CuSP gene is selected from the group consisting of CuSP-1 having a genomic nucleotide sequence as set forth in SEQ ID NO:1 or a functional variant or homologue thereof, CuSP-2 having a genomic nucleotide sequence as set forth in SEQ ID NO:89 or a functional variant or homologue thereof, CuSP-3 having a genomic nucleotide sequence as set forth in SEQ ID NO: 167 or a functional variant or homologue thereof and any combination thereof.

According to a further embodiment of the present invention, the functional variant or homologue has at least 75% sequence identity to said CuSP nucleotide sequence.

It is within the scope of the present invention that the modified cucumber plant comprises at least one genetic modification introduced in said at least one CuSP gene using targeted genome modification.

It is further within the scope of the present invention to disclose that the genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO:4-88, 92-166, 170-255 and any combination thereof.

It is further within the scope of the present invention to provide a plant part, plant cell, plant fruit or plant seed of a modified cucumber plant as defined in any of the above, wherein said plant part, plant cell, plant fruit or plant seed comprises at least one genetic modification conferring reduced expression of at least one Cucumber SELF PRUNING (SP) (CuSP) gene.

According to a further embodiment, the present invention provides a method for producing a modified Cucumber plant exhibiting at least one improved domestication trait, wherein said method comprises steps of genetically modifying at least one Cucumber SELF PRUNING (SP) (CuSP) gene.

According to a further embodiment, the present invention provides an isolated nucleotide sequence having at least 75% sequence identity to a CuSP genomic nucleotide sequence selected from the group consisting of SEQ ID NO:1, SEQ ID NO:89 and SEQ ID NO:167.

According to a further embodiment, the present invention provides an isolated nucleotide sequence having at least 75% sequence identity to a CuSP nucleotide coding sequence selected from the group consisting of SEQ ID NO:2, SEQ ID NO:90 and SEQ ID NO:168.

According to a further embodiment, the present invention provides an isolated amino acid sequence having at least 75% sequence similarity to a CuSP amino acid sequence selected from the group consisting of SEQ ID NO:3, SEQ ID NO:91 and SEQ ID NO:169.

According to a further embodiment, the present invention provides an isolated nucleotide sequence having at least 75% sequence identity to a CuSP-targeted gRNA nucleotide sequence as set forth in SEQ ID NO:4-88, 92-166 and 170-255.

According to a further embodiment, the present invention provides a use of a nucleotide sequence as set forth in at least one of SEQ ID NO:4-88 and any combination thereof, SEQ ID NO:92-166 and any combination thereof, and SEQ ID NO:170-255 and any combination thereof, for targeted genome modification of Cucumber SP-1 (CuSP-1), Cucumber SP-2 (CuSP-2), and Cucumber SP-3 (CuSP-3) allele or gene, respectively.

As used herein the term “about” denotes ±25% of the defined amount or measure or value. As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same Cucumber species or strain or variety or to sibling plant, or one or more individuals having one or both parents in common. The term “corresponding” further encompass a wild type cucumber plant or a cucumber plant lacking a genetic modification conferring reduced expression of at least one Cucumber SELF PRUNING (SP) (CuSP) gene (also used herein as wild type or non-modified cucumber plant), or a cucumber plant lacking the improved domestication or agronomic trait.

According to further aspects of the current invention, the term “corresponding” or “corresponding to position” as used herein, refers in the context of the present invention to sequence homology or sequence identity. These terms relate to two or more nucleic acid or protein sequences, that are the same or have a specified percentage of amino acid residues or nucleotides that are the same, when compared and aligned for maximum correspondence, as measured using one of the available sequence comparison algorithms or by visual inspection. If two sequences, which are to be compared with each other, differ in length, sequence identity preferably relates to the percentage of the nucleotide residues of the shorter sequence, which are identical with the nucleotide residues of the longer sequence. As used herein, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. According to further aspects of the invention, the term “corresponding to the nucleotide sequence” or “corresponding to position”, refers to variants, homologues and fragments of the indicated nucleotide sequence, which possess or perform the same biological function or correlates with the same phenotypic characteristic of the indicated nucleotide sequence.

Another indication that two nucleic acid sequences are substantially identical or that a sequence is “corresponding to the nucleotide sequence” is that the two molecules hybridize to each other under stringent conditions. High stringency conditions, such as high hybridization temperature and low salt in hybridization buffers, permits only hybridization between nucleic acid sequences that are highly similar, whereas low stringency conditions, such as lower temperature and high salt, allows hybridization when the sequences are less similar.

In other embodiments of the invention, such substantially identical sequences refer to polynucleotide or amino acid sequences that share at least about 80% similarity, preferably at least about 90% similarity, alternatively, about 95%, 96%, 97%, 98% or 99% similarity to the indicated polynucleotide or amino acid sequences.

According to other aspects of the invention, the term “corresponding” refers also to complementary sequences or base pairing such that when they are aligned antiparallel to each other, the nucleotide bases at each position in the sequences will be complementary. The degree of complementarity between two nucleic acid strands may vary.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit (e.g. cucumber fruit), flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits (e.g. cucumber fruit, particularly modified cucumber fruit as disclosed by the current invention), pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. loss of function mutation in at least one CuSP gene.

The term “Cucumber” refers hereinafter to a genus of flowering plants in the family Cucurbitaceae. In certain aspects of the present invention it refers a plant species within the genus Cucumis, such as the species C. sativus.

The term “domestication” or “domestication trait” as used herein refers to any agronomic trait desirable for crops or crop cultivation. It is herein acknowledged that based on relationships between wild progenitors and domesticated descendants domestication phenotype had been selected in crop species.

One of the traits that appeared during domestication of common crops is determinacy, in which stems end with a terminal inflorescence. It is further within the scope of the present invention that domestication refers to a selection process conducted by humans among wild plants for adaptation to human cultivation and consumption. This selection process has brought about marked changes in the morphology and physiology of crop plants. One of the traits selected during crop domestication is a more compact growth habit, manifested by a series of traits such as reduced branching, shorter internodes, fewer nodes, reduced twining and, in some cases, a determinate stem ending. The wild relatives are generally viny, herbaceous plants, with a high level of branching, many nodes, long and twining internodes, and diageotropic branch growth. The vininess allows plants to compete with surrounding plants for light in the shrubby or arboreal vegetation in which these wild plants grow naturally. Determinacy is, therefore, a trait selected during or after domestication. Thus, after domestication, generally, stems have a finite length, and flowering occurs earlier than in indeterminate types. Since the determinate growth habit allows mechanical harvesting with a shorter growing cycle, cultivars with the determinacy trait are preferred in several crop species, such as Cucumber.

It is noted that crops originating from their wild ancestors through domestication, during which artificial selection acts as a powerful driver, has modified crop genomes as well as modified morphological characteristics and growth habits beneficial to humans.

It is emphasized that the genetic base of cucumber, an economically important vegetable crop, has become extraordinarily narrow due to recurrent use of limited variation during breeding. Most cucumber cultivars have indeterminate plant habit, where the stem elongates continuously, and 1-2 primary lateral branches originating from the main stem. Some cultivars also produce secondary lateral branches (originating from primary lateral branches) under some growing conditions, which is under polygenic control. More branching occurs when plants are grown at low density. The current invention solves this problem by providing Cucumber plant with improved domestication and/or agronomical trait, particularly by using gene editing technique.

The term ‘SELF-PRUNING’ or ‘SP’ in the context of the present invention refers to a gene which encodes a flowering repressor that modulates sympodial growth. It is herein shown that mutations in the SP orthologue cause an acceleration of sympodial cycling and shoot termination. It is further acknowledged that the SELF PRUNING (SP) gene controls the regularity of the vegetative-reproductive switch along the compound shoot of, for example, tomato and thus conditions the ‘determinate’ (sp/sp) and ‘indeterminate’ (SP) growth habits of the plant. SP is a developmental regulator which is homologous to CENTRORADIALIS (CEN) from Antirrhinum and TERMINAL FLOWER 1 (TFL1) and FLOWERING LOCUS T (FT) from Arabidopsis.

The present invention discloses that SP is a member of a gene family in Cucumber composed of at least three genes. The Cucumber SP genes comprise CuSP-1, CuSP-2 and CuSP-3, encoded by genomic sequence as set forth in SEQ. ID. NO: 1, 89 and 167, coding sequence as set forth in SEQ. ID. NO:2, 90 and 168, and amino acid sequence as set forth in SEQ. ID. NO:3, 91 and 169, respectively. According to main aspects of the present invention, genome editing-targeted mutation in at least one of the aforementioned CuSP genes, which reduces the functional expression of the gene, affect the plant sympodial growth habit which plays a key role in determining plant architecture.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cucumber plants with improved domestication traits are generated using genome editing mechanism. This technique enables to achieve in planta modification of specific genes that relate to and/or control the flowering time and plant architecture in the Cucumber plant.

The term “genome editing”, or “genome/genetic modification” or “genome engineering” or “gene editing” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

Cas = CRISPR-associated genes
Cas9, Csn1 = a CRISPR-associated protein containing two nuclease
domains, that is programmed by small RNAs to cleave DNA
crRNA = CRISPR RNA
dCAS9 = nuclease-deficient Cas9
DSB = Double-Stranded Break
gRNA = guide RNA
HDR = Homology-Directed Repair
HNH = an endonuclease domain named for characteristic histidine
and asparagine residues
Indel = insertion and/or deletion
NHEJ = Non-Homologous End Joining
PAM = Protospacer-Adjacent Motif
RuvC = an endonuclease domain named for an E. coli protein involved
in DNA repair
sgRNA = single guide RNA
tracrRNA, trRNA = trans-activating crRNA
TALEN = Transcription-Activator Like Effector Nuclease
ZFN = Zinc-Finger Nuclease

According to specific aspects of the present invention, the CRISPR (Clustered Regularly Interspaced Short Palindromic Repeats) and CRISPR-associated (Cas) genes are used for the first time for generating genome modification in targeted genes in the Cucumber plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′—of the crRNA complementary sequence.

According to some embodiments, the gRNA sequence comprises a 3′ NGG Protospacer Adjacent Motif (PAM) selected from the group consisting of NGG (SpCas), NNNNGATT (NmeCas9), NNAGAAW, (StCas9), NAAAAC (TdCas9), NNGRRT (SaCas9) and TBN (Cas-phi).

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

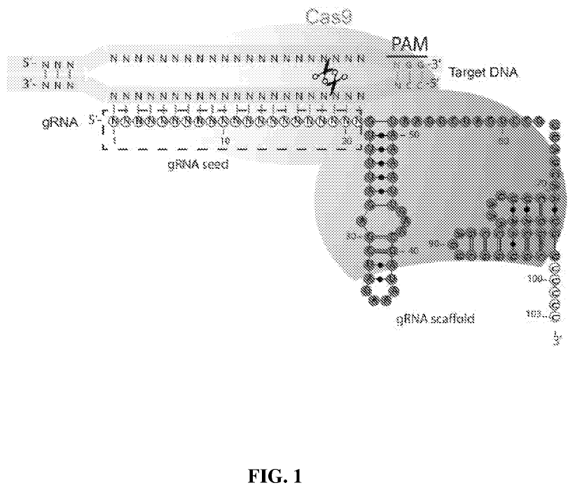

Reference is now made to FIG. 1 schematically presenting an example of CRISPR/Cas9 mechanism of action as depicted by Xie, Kabin, and Yinong Yang. “RNA-guided genome editing in plants using a CRISPR-Cas system.” Molecular plant 6.6 (2013): 1975-1983. As shown in this figure, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells.

More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term ‘sympodial growth’ as used herein refers to a type of bifurcating branching pattern where one branch develops more strongly than the other, resulting in the stronger branches forming the primary shoot and the weaker branches appearing laterally. A sympodium, also referred to as a sympode or pseudaxis, is the primary shoot, comprising the stronger branches, formed during sympodial growth. In some aspects of the present invention, sympodial growth occurs when the apical meristem is terminated and growth is continued by one or more lateral meristems, which repeat the process. The apical meristem may be consumed to make an inflorescence or other determinate structure, or it may be aborted.

It is further within the scope of the current invention that the shoot section between two successive inflorescences is called the ‘sympodium’, and the number of leaf nodes per sympodium is referred to as the ‘sympodial index’ (spi). The first termination event activates the ‘sympodial cycle’. In sympodial plants, the apparent main shoot consists of a reiterated array of ‘sympodial units’. A mutant sp gene accelerates the termination of sympodial units but does not change the sympodial habit. The result is a progressive reduction in the number of vegetative nodes between inflorescences in a pattern that depends on light intensity and genetic background.

The term “earliness” refers hereinafter to early flowering and/or rapid transition from the vegetative to reproductive stages, or reduced ‘time to initiation of flowering’ and more generally to earlier completion of the life-cycle.

The term ‘reduced flowering time’ as used herein refers to time to production of first inflorescence. Such a trait can be evaluated or measured, for example, with reference to the number of leaves produced prior to appearance of the first inflorescence.

The term ‘harvest index’ can be herein defined as the total yield per plant weight.

The term ‘day length’ or ‘day length sensitivity’ as used in the context of the present invention generally refers to photoperiodism, which is the physiological reaction of organisms to the length of day or night. Photoperiodism can also be defined as the developmental responses of plants to the relative lengths of light and dark periods. Plants are classified under three groups according to the photoperiods: short-day plants, long-day plants, and day-neutral plants. Photoperiodism affects flowering by inducing the shoot to produce floral buds instead of leaves and lateral buds. It is within the scope of the present invention that Cucumber is included within the short-day facultative plants. The Cucumber plants of the present invention are genetically modified so as to exhibit loss of day-length sensitivity, which is highly desirable agronomic trait enabling enhanced yield of the cultivated crop.

The term ‘determinate’ or ‘determinate growth’ as used herein refers to plant growth in which the main stem ends in an inflorescence or other reproductive structure (e.g. a bud) and stops continuing to elongate indefinitely with only branches from the main stem having further and similarly restricted growth. It also refers to growth characterized by sequential flowering from the central or uppermost bud to the lateral or basal buds. It further means naturally self-limited growth, resulting in a plant of a definite maximum size.

The term ‘indeterminate’ or ‘indeterminate growth’ as used herein refers to plant growth in which the main stem continues to elongate indefinitely without being limited by a terminal inflorescence or other reproductive structure. It also refers to growth characterized by sequential flowering from the lateral or basal buds to the central or uppermost buds.

The term “orthologue” as used herein refers hereinafter to one of two or more homologous gene sequences found in different species.

The term “functional variant” or “functional variant of a nucleic acid or amino acid sequence” as used herein, for example with reference to SEQ ID NOs: 1, 4 or 7 refers to a variant of a sequence or part of a sequence which retains the biological function of the full non-variant allele (e.g. CuSP allele) and hence has the activity of SP expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the three identified SP genes in Cucumber, namely CuSP-1, CuSP-2 and CuSP-3 having the genomic nucleotide sequence as set forth in SEQ ID NOs: 1, 89 and 167, respectively.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Cucumber plants of the present invention comprise homozygous configuration of at least one of the mutated Cusp genes (i.e. Cusp-1, Cusp-2 and Cusp-3).

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context.

Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant. Specifically, the endogenous nucleic acid sequences of each of the SP homologs in Cucumber (nucleic acid sequences CuSP-1, CuSP-2 and CuSP-3) have been altered compared to wild type sequences using mutagenesis and/or genome editing methods as described herein. This causes inactivation of the endogenous SP gene and thus disables SP function. Such plants have an altered phenotype and show improved domestication traits such as determinant plant architecture, synchronous and/or early flowering and loss of day length sensitivity compared to wild type plants. Therefore, the improved domestication phenotype is conferred by the presence of at least one mutated endogenous Cusp gene in the Cucumber plant genome which has been specifically targeted using genome editing technique.

According to further aspects of the present invention, the at least one improved domestication trait is not conferred by the presence of transgenes expressed in Cucumber.

It is further within the scope of the current invention that sp mutations that down-regulate or disrupt functional expression of the wild-type SP sequence, may be recessive, such that they are complemented by expression of a wild-type sequence.

It is further noted that a wild type Cucumber plant is a plant that does not have any mutant sp alleles.

Main aspects of the invention involve targeted mutagenesis methods, specifically genome editing, and exclude embodiments that are solely based on generating plants by traditional breeding methods. In a further embodiment of the current invention, as explained herein, the improved domestication at least one trait is not due to the presence of a transgene.

The inventors have generated mutant Cucumber lines with mutations inactivating at least one CuSP homoeoallele which confer heritable improved domestication trait(s). In this way no functional CuSP protein is made. Thus, the invention relates to these mutant Cucumber lines and related methods.

It is further within the scope of the present invention that breeding Cucumber cultivars with mutated sp allele enables the mechanical harvest of the plant. According to a further aspect of the present invention, loss of SP function results in compact Cucumber plants with reduced height, reduced number of sympodial units and determinate growth when compared with WT Cucumber.

According to a main aspect of the present invention, modifying Cucumber shoot architecture by selection for mutations in florigen flowering pathway genes allowed major improvements in plant architecture and yield. In particular, a mutation in the antiflorigen SELFPRUNING (SP) gene (sp classic) provided compact ‘determinate’ growth that translated to a burst of flowers, thereby enabling largescale field production.

The work inter alia described has important implications. The results have shown that CRISPR/Cas9 can be used to create heritable mutations in florigen pathway family members that result in desirable phenotypic effects.

To edit multiple domestication genes simultaneously and stack the resulting allelic variants, on option is that several gRNAs can be assembled to edit several genes into one construct, by using the Csy4 multi-gRNA system. The construct is then transformed via an appropriate vector into several wild-Cucumber accessions.

It is further within the scope of the current invention that Cucumber SP genes, namely CuSP-1, CuSP-2 and CuSP-3 having genomic nucleotide sequence as set forth in SEQ. ID. NO: 1, 89 and 167, coding sequence as set forth in SEQ. ID. NO:2, 90 and 168, and amino acid sequence as set forth in SEQ. ID. NO:3, 91 and 169, respectively, were targeted using guide RNAs. Several mutated alleles have been identified. Notably, the plants with mutated sp alleles were more compact than the wild type plants lacking the mutated allele.

The loss of function mutation may be a deletion or insertion (“indels”) with reference the wild type CuSP allele sequence. The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants of a particular CuSP nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant CuSP nucleotide sequence of the CuSP allele as shown in SEQ ID NO 1, 89 or 167. Sequence alignment programs to determine sequence identity are well known in the art.

Also, the various aspects of the invention encompass not only a CuSP nucleic acid sequence or amino acid sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, in this case improved domestication trait.

According to a further embodiment of the invention, the herein newly identified Cucumber SP (CuSP) have been targeted using the double sgRNA strategy.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion.

It is within the scope of the present invention that the usage of CRISPR/Cas system for the generation of Cucumber plants with at least one improved domestication trait, allows the modification of predetermined specific DNA sequences without introducing foreign DNA into the genome by GMO techniques. According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence (for example see FIG. 1). The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that improved domestication traits in Cucumber plants is herein produced by generating gRNA with homology to a specific site of predetermined genes in the Cucumber genome i.e. SP gene, sub cloning this gRNA into a plasmid containing the Cas9 gene, and insertion of the plasmid into the Cucumber plant cells. In this way site specific mutations in the SP genes are generated thus effectively creating non-active molecules, resulting in determinant growth habit of the genome edited plant.

According to one embodiment, the present invention provides a modified Cucumber plant exhibiting at least one improved domestication trait, wherein said modified plant comprises at least one genetic modification conferring reduced expression of at least one Cucumber SELF PRUNING (SP) (CuSP) gene.

According to a further embodiment of the present invention, the CuSP gene is selected from the group consisting of CuSP-1 having a genomic nucleotide sequence as set forth in SEQ ID NO:1 or a functional variant or homologue thereof, CuSP-2 having a genomic nucleotide sequence as set forth in SEQ ID NO:89 or a functional variant or homologue thereof, CuSP-3 having a genomic nucleotide sequence as set forth in SEQ ID NO: 167 or a functional variant or homologue thereof and any combination thereof.

According to a further embodiment of the present invention, the functional variant or homologue has at least 75% sequence identity to said CuSP nucleotide sequence.

According to a further embodiment of the present invention, the modified cucumber plant exhibits at least one improved domestication trait as compared to a corresponding Cucumber plant lacking said genetic modification.

According to a further embodiment of the present invention, the genetic modification is introduced using mutagenesis, small interfering RNA (siRNA), microRNA (miRNA), artificial miRNA (amiRNA), DNA introgression, endonucleases or any combination thereof.

According to a further embodiment of the present invention, the modified cucumber plant comprises at least one genetic modification introduced in said at least one CuSP gene using targeted genome modification.

According to a further embodiment of the present invention, the genetic modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

According to a further embodiment of the present invention, the Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

According to a further embodiment of the present invention, the genetically modified CuSP gene is a CRISPR/Cas9—induced heritable mutated allele.

According to a further embodiment of the present invention, the genetic modification is a missense mutation, nonsense mutation, insertion, deletion, indel, substitution or duplication.

According to a further embodiment of the present invention, the insertion or the deletion produces a gene comprising a frameshift.

According to a further embodiment of the present invention, the plant is homozygous for said at least one genetically modified CuSP gene.

According to a further embodiment of the present invention, the genetic modification is in the coding region of said gene, a mutation in the regulatory region of said gene, or an epigenetic factor.

According to a further embodiment of the present invention, the genetic modification is a silencing mutation, a knockdown mutation, a knockout mutation, a loss of function mutation or any combination thereof.

According to a further embodiment of the present invention, the genetic modification is generated in planta.

According to a further embodiment of the present invention, the genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO:4-88, 92-166, 170-255 and any combination thereof.

According to a further embodiment of the present invention, the genetic modification in said CuSP-1 is generated in planta via introduction of a construct comprising (a) Cas DNA and gRNA sequence selected from the group consisting of SEQ ID NO:4-88 and any combination thereof, or (b) a ribonucleoprotein (RNP) complex comprising Cas protein and gRNA sequence selected from the group consisting of SEQ ID NO:4-88 and any combination thereof.

According to a further embodiment of the present invention, the mutation in said CuSP-2 is generated in planta via introduction of a construct comprising (a) Cas DNA and gRNA sequence selected from the group consisting of SEQ ID NO:92-166 and any combination thereof, or (b) a ribonucleoprotein (RNP) complex comprising Cas protein and gRNA sequence selected from the group consisting of SEQ ID NO:92-166 and any combination thereof.

According to a further embodiment of the present invention, the mutation in said CuSP-3 is generated in planta via introduction of a construct comprising (a) Cas DNA and gRNA sequence selected from the group consisting of SEQ ID NO:170-SEQ ID NO:255 and any combination thereof, or (b) a ribonucleoprotein (RNP) complex comprising Cas protein and gRNA sequence selected from the group consisting of SEQ ID NO: 170-255 and any combination thereof.

According to a further embodiment of the present invention, the gRNA sequence comprises a 3′ NGG Protospacer Adjacent Motif (PAM).

According to a further embodiment of the present invention, the construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

According to a further embodiment of the present invention, the plant has decreased expression levels of at least one of said CuSP genes.

According to a further embodiment of the present invention, the sequence of said expressed CuSP gene is selected from the group consisting of: SEQ ID NO:2, SEQ ID NO:3, SEQ ID NO:90, SEQ ID NO:91, SEQ ID NO: 168 and SEQ ID NO:169 or a functional variant or homologue thereof.

According to a further embodiment of the present invention, the plant is semi-determinant.

According to a further embodiment of the present invention, the plant has determinant growth habit.

According to a further embodiment of the present invention, the plant flowers earlier than a corresponding Cucumber plant lacking said genetic modification.

According to a further embodiment of the present invention, the plant exhibits improved earliness as compared to a corresponding Cucumber plant lacking said genetic modification.

According to a further embodiment of the present invention, the plant exhibits suppressed sympodial shoot termination as compared to a corresponding Cucumber plant lacking said genetic modification.

According to a further embodiment of the present invention, the plant exhibits similar sympodial shoot termination as compared to a corresponding Cucumber plant lacking said genetic modification.

According to a further embodiment of the present invention, the domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is further within the scope of the current invention to disclose a Cucumber plant, plant part, plant fruit or plant cell as defined in any of the above, wherein said plant does not comprise a transgene.

It is further within the scope of the current invention to disclose a plant part, plant cell, plant fruit or plant seed of a modified cucumber plant as defined in any of the above, wherein said plant part, plant cell, plant fruit or plant seed comprises at least one genetic modification conferring reduced expression of at least one Cucumber SELF PRUNING (SP) (CuSP) gene.

It is further within the scope of the current invention to disclose a tissue culture of regenerable cells, protoplasts or callus obtained from the modified Cucumber plant as defined in any of the above.

According to a further embodiment of the present invention, the modified plant genotype is obtainable by deposit under accession number with NCIMB Aberdeen AB21 9YA, Scotland, UK or with ATCC.

According to a further embodiment, the present invention provides a method for producing a modified Cucumber plant exhibiting at least one improved domestication trait, wherein said method comprises steps of genetically modifying at least one Cucumber SELF PRUNING (SP) (CuSP) gene.

It is further within the scope of the current invention to disclose the method as defined in any of the above, comprises steps of producing the modified Cucumber plant using targeted genome modification, by genetically introducing a loss of function mutation in said at least one Cucumber SELF PRUNING (SP) (CuSP) gene.

It is further within the scope of the current invention to disclose the method as defined in any of the above, wherein said genetic modification confers reduced expression of at least one Cucumber SELF PRUNING (SP) (CuSP) gene.

It is further within the scope of the current invention to disclose the method as defined in any of the above, wherein said modified cucumber plant exhibits at least one improved domestication trait as compared to a corresponding Cucumber plant lacking said genetic modification.

It is further within the scope of the current invention to disclose the method as defined in any of the above, wherein said method comprises steps of: (a) identifying at least one Cucumber SP (CuSP) gene or allele; (b) synthetizing at least one guide RNA (gRNA) comprising a nucleotide sequence complementary to said at least one identified CuSP allele; (c) transforming Cucumber plant cells with a construct comprising (a) Cas nucleotide sequence operably linked to said at least one gRNA, or (b) a ribonucleoprotein (RNP) complex comprising Cas protein and said at least one gRNA; (d) screening the genome of said transformed plant cells for induced targeted loss of function mutation in at least one of said CuSP allele or gene; (e) regenerating Cucumber plant carrying said loss of function mutation in at least one of said CuSP allele or gene; and (f) screening said regenerated plants for a Cucumber plant with improved domestication trait.

It is further within the scope of the current invention to disclose the method as defined in any of the above, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said CuSP allele or gene.

It is further within the scope of the current invention to disclose the method as defined in any of the above, wherein said CuSP Cucumber gene is selected from the group consisting of CuSP-1 having a genomic nucleotide sequence as set forth in SEQ ID NO:1 or a functional variant or homologue thereof, CuSP-2 having a genomic nucleotide sequence as set forth in SEQ ID NO:89 or a functional variant or homologue thereof, CuSP-3 having a genomic nucleotide sequence as set forth in SEQ ID NO: 167 or a functional variant or homologue thereof and any combination thereof.

It is further within the scope of the current invention to disclose the method as defined in any of the above, wherein said functional variant or homologue has at least 75% sequence identity to said CuSP nucleotide sequence.

It is further within the scope of the current invention to disclose the method as defined in any of the above, wherein said genetic modification is introduced using mutagenesis, small interfering RNA (siRNA), microRNA (miRNA), artificial miRNA (amiRNA), DNA introgression, endonucleases or any combination thereof.

It is further within the scope of the current invention to disclose the method as defined in any of the above, wherein said genetic modification is introduced using targeted gene editing.

It is further within the scope of the current invention to disclose the method as defined in any of the above, wherein said genetic modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is further within the scope of the current invention to disclose the method as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

It is further within the scope of the current invention to disclose the method as defined in any of the above, wherein the mutated CuSP gene is a CRISPR/Cas9—induced heritable mutated allele or gene.

It is further within the scope of the current invention to disclose the method as defined in any of the above, wherein said genetic modification is a missense mutation, nonsense mutation, insertion, deletion, indel, substitution or duplication.

It is further within the scope of the current invention to disclose the method as defined in any of the above, wherein the insertion or the deletion produces a gene comprising a frameshift.

It is further within the scope of the current invention to disclose the method as defined in any of the above, wherein said modified plant is homozygous for said at least one CuSP mutated gene.

It is further within the scope of the current invention to disclose the method as defined in any of the above, wherein said genetic modification is in the coding region of said gene, a mutation in the regulatory region of said gene, or an epigenetic factor.

It is further within the scope of the current invention to disclose the method as defined in any of the above, wherein said genetic modification is a silencing mutation, a knockdown mutation, a knockout mutation, a loss of function mutation or any combination thereof.

It is further within the scope of the current invention to disclose the method as defined in any of the above, wherein said genetic modification is generated in planta.

It is further within the scope of the current invention to disclose the method as defined in any of the above, wherein said genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO: 4-88, 92-166, 170-255 and any combination thereof.

It is further within the scope of the current invention to disclose the method as defined in any of the above, wherein said mutation in said CuSP-1 is generated in planta via introduction of a construct comprising (a) Cas DNA and gRNA sequence selected from the group consisting of SEQ ID NO:4-88 and any combination thereof, or (b) a ribonucleoprotein (RNP) complex comprising Cas protein and gRNA sequence selected from the group consisting of SEQ ID NO:4-88 and any combination thereof.

It is further within the scope of the current invention to disclose the method as defined in any of the above, wherein said mutation in said CuSP-2 is generated in planta via introduction of a construct comprising (a) Cas DNA and gRNA sequence selected from the group consisting of SEQ ID NO:92-166 and any combination thereof, or (b) a ribonucleoprotein (RNP) complex comprising Cas protein and gRNA sequence selected from the group consisting of SEQ ID NO:92-166 and any combination thereof.

It is further within the scope of the current invention to disclose the method as defined in any of the above, wherein said mutation in said CuSP-3 is generated in planta via introduction of a construct comprising (a) Cas DNA and gRNA sequence selected from the group consisting of SEQ ID NO: 170-255 and any combination thereof, or (b) a ribonucleoprotein (RNP) complex comprising Cas protein and gRNA sequence selected from the group consisting of SEQ ID NO:170-255 and any combination thereof.

It is further within the scope of the current invention to disclose the method as defined in any of the above, wherein said gRNA sequence comprises a 3′ NGG Protospacer Adjacent Motif (PAM).

It is further within the scope of the current invention to disclose the method as defined in any of the above, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is further within the scope of the current invention to disclose the method as defined in any of the above, wherein said modified plant has decreased expression levels of at least one of said CuSP genes.

It is further within the scope of the current invention to disclose the method as defined in any of the above, wherein the sequence of said expressed CuSP gene is selected from the group consisting of: SEQ ID NO:2, SEQ ID NO:3, SEQ ID NO:90, SEQ ID NO:91, SEQ ID NO:168 and SEQ ID NO:169 or a functional variant or homologue thereof.

It is further within the scope of the current invention to disclose the method as defined in any of the above, wherein said modified plant is semi-determinant.

It is further within the scope of the current invention to disclose the method as defined in any of the above, wherein said modified plant has determinant growth habit.

It is further within the scope of the current invention to disclose the method as defined in any of the above, wherein said modified plant flowers earlier than a corresponding Cucumber plant lacking said genetic modification.

It is further within the scope of the current invention to disclose the method as defined in any of the above, wherein said modified plant exhibits improved earliness as compared to a corresponding Cucumber plant lacking said genetic modification.

It is further within the scope of the current invention to disclose the method as defined in any of the above, wherein said modified plant exhibits suppressed sympodial shoot termination as compared to a corresponding Cucumber plant lacking said genetic modification.

It is further within the scope of the current invention to disclose the method as defined in any of the above, wherein said modified plant exhibits similar sympodial shoot termination as compared to a corresponding Cucumber plant lacking said genetic modification.

It is further within the scope of the current invention to disclose the method as defined in any of the above, wherein said modified plant exhibits suppressed or reduced day-length sensitivity as compared to a corresponding Cucumber plant lacking said genetic modification.

It is further within the scope of the current invention to disclose a modified Cucumber plant, plant part, plant fruit or plant cell produced by the method as defined in any of the above, wherein said plant does not comprise a transgene.

It is further within the scope of the current invention to disclose a plant part, plant cell, plant fruit or plant seed of a plant produced by the method as defined in any of the above.

It is further within the scope of the current invention to disclose a tissue culture of regenerable cells, protoplasts or callus obtained from the modified Cucumber plant produced by the method as defined in any of the above.

It is further within the scope of the current invention to disclose the method as defined in any of the above, wherein said plant genotype is obtainable by deposit under accession number with NCIMB Aberdeen AB21 9YA, Scotland, UK or with ATCC.

It is further within the scope of the current invention to disclose the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

According to a further embodiment, the present invention provides an isolated nucleotide sequence having at least 75% sequence identity to a CuSP genomic nucleotide sequence selected from the group consisting of SEQ ID NO:1, SEQ ID NO:89 and SEQ ID NO:167.

According to a further embodiment of the present invention, the isolated nucleotide sequence having at least 75% sequence identity to a CuSP nucleotide coding sequence selected from the group consisting of SEQ ID NO:2, SEQ ID NO:90 and SEQ ID NO: 168.

According to a further embodiment, the present invention provides an isolated amino acid sequence having at least 75% sequence similarity to a CuSP amino acid sequence selected from the group consisting of SEQ ID NO:3, SEQ ID NO:91 and SEQ ID NO:169.

According to a further embodiment, the present invention provides an isolated nucleotide sequence having at least 75% sequence identity to a CuSP-targeted gRNA nucleotide sequence as set forth in SEQ ID NO:4-88, 92-166 and 170-255.

According to a further embodiment, the present invention provides a use of a nucleotide sequence having at least 75% sequence identity to a nucleic acid sequence as set forth in at least one of SEQ ID NO:4-88 and any combination thereof for targeted genome modification of Cucumber SP-1 (CuSP-1) allele or gene.

It is further within the scope of the current invention to disclose a use of a nucleotide sequence having at least 75% sequence identity to a nucleic acid sequence as set forth in at least one of SEQ ID NO:92-166 and any combination thereof for targeted genome modification of Cucumber SP-2 (CuSP-2) allele or gene.

It is further within the scope of the current invention to disclose a use of a nucleotide sequence having at least 75% sequence identity to a nucleic acid sequence as set forth in at least one of SEQ ID NO: 170-255 and any combination thereof for targeted genome modification of Cucumber SP-3 (CuSP-3) allele or gene.

In order to understand the invention and to see how it may be implemented in practice, a plurality of preferred embodiments will now be described, by way of non-limiting example only, with reference to the following examples.

### Example 1

Production of Cucumber Plants with Improved Domestication Traits by Targeted Gene Editing

Production of Cucumber lines with mutated sp gene may be achieved by at least one of the following breeding/cultivation schemes:

Scheme 1:
line stabilization by self pollination
Generation of F6 parental lines
Genome editing of parental lines
Crossing edited parental lines to generate an F1 hybrid plant

Scheme 2:
Identifying genes/alleles of interest
Designing gRNA
Transformation of plants with Cas9+gRNA constructs
Screening and identifying editing events
Genome editing of parental lines

It is noted that line stabilization may be performed by the following:
Induction of male flowering on plants
Self pollination

According to some embodiments of the present invention, line stabilization requires about 6 self-crossing (6 generations) and done through a single seed descent (SSD) approach.

F1 hybrid seed production: Novel hybrids are produced by crosses between different Cucumber strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Cucumber are developed and provided by the current invention:
Genotyping markers—germplasm used in the current invention is genotyped using molecular markers, in order to allow a more efficient breeding process and identification of the SP editing event(s).

It is further within the scope of the current invention that allele and genetic variation is analyzed for the Cucumber strains used.

Reference is now made to optional stages that have been used for the production of mutated SP Cucumber plants by genome editing:

Stage 1: Identifying Cucumis sativus (C. sativus) SP genes (CuSP).

Three SP orthologues have herein been identified in Cucumis sativus (C. sativus) namely CuSP-1, CuSP-2 and CuSP-3. These homologous genes have been sequenced and mapped.

CuSP-1 has been mapped to CsGy3G032260:29750603-29747435 [Chr3, CsGy3G032260 (gene) Cucumber (Gy14) v2] and has a genomic sequence as set forth in SEQ ID NO:1. The CuSP-1 gene has a coding sequence as set forth in SEQ ID NO:2 and it encodes an amino acid sequence as set forth in SEQ ID NO:3.

CuSP-2 has been mapped to CsGy6G024900:21554140-21555525 [Chr6, CsGy6G024900 (gene) Cucumber (Gy14) v2] and has a genomic sequence as set forth in SEQ ID NO:89. The CuSP-2 gene has a coding sequence as set forth in SEQ ID NO:90, and it encodes an amino acid sequence as set forth in SEQ ID NO:91.

CuSP-3 has been mapped to CsGy6G012560:10805149-10806778 [Chr6, CsGy6G012560 (gene) Cucumber (Gy14) v2] and has a genomic sequence as set forth in SEQ ID NO:167. The CuSP-3 gene has a coding sequence as set forth in SEQ ID NO: 168, and it encodes an amino acid sequence as set forth in SEQ ID NO:169.

Stage 2: Designing and synthesizing gRNA molecules corresponding to the sequence targeted for editing, i.e. sequences of each of the genes CuSP-1, CuSP-2 and CuSP-3. It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different SP homologues or alleles and for different Cucumber strains.

The designed gRNA molecules were cloned into suitable vectors and their sequence has been verified. In addition, different Cas9 versions have been analyzed for optimal compatibility between the Cas9 protein activity and the gRNA molecule in the Cucumber plant.

Reference is now made to Tables 1-3 presenting gRNA sequences constructed for silencing CuSP-1, CuSP-2 and CuSP-3 genes, respectively. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system. The genomic DNA sense strand is marked as “1”, and the antisense strand is marked as “−1”.

TABLE 1
gRNA and PAM sequences targeted for CuSP-1

| | Position in | | |
|---|---|---|---|
| SEQ ID | SEQ ID | | |
| NO | NO: 1 | Sequence | PAM |
| 4 | 73 | TCTGAGTGCTTTTTGAGAAA | TGG |
| 5 | 103 | ACCAACAACAAGAGGTTCTG | AGG |
| 6 | 111 | ATCACTCTACCAACAACAAG | AGG |
| 7 | 113 | TCCTCAGAACCTCTTGTTGT | TGG |
| 8 | 125 | CTTGTTGTTGGTAGAGTGAT | TGG |
| 9 | 151 | CATCTTCATGCTTTGTGTGA | AGG |
| 10 | 200 | AATAATAAGCAAGTTTTCAA | TGG |
| 11 | 212 | CAGCAGAAGGAAAGAACTCA | TGG |
| 12 | 225 | GGTTTAGCAGCAACAGCAGA | AGG |
| 13 | 240 | CTGCTGTTGCTGCTAAACCT | AGG |
| 14 | 241 | TGCTGTTGCTGCTAAACCTA | GGG |
| 15 | 246 | CCACCATGGATTTCAGCCCT | AGG |
| 16 | 254 | AAACCTAGGGCTGAAATCCA | TGG |
| 17 | 257 | CCTAGGGCTGAAATCCATGG | TGG |
| 18 | 260 | AAGATCTCAGGTCACCACCA | TGG |
| 19 | 272 | CCAGAGTGAAGAAAGATCTC | AGG |
| 20 | 283 | CCTGAGATCTTTCTTCACTC | TGG |
| 21 | 319 | AAGTTTGAGATAAACAAACA | AGG |
| 22 | 355 | TACAAAAAATGATTTATCTG | AGG |
| 23 | 360 | AAAATGATTTATCTGAGGCT | TGG |
| 24 | 397 | TGCATGTTTTGAGTTGTGTG | TGG |
| 25 | 457 | ATATTGATTATATCGTTCAT | AGG |
| 26 | 517 | AAGAAAAAAATTAAAAAAAG | AGG |
| 27 | 518 | AGAAAAAAATTAAAAAAAGA | GGG |
| 28 | 519 | GAAAAAAATTAAAAAAAGAG | GGG |
| 29 | 520 | AAAAAAATTAAAAAAAGAGG | GGG |
| 30 | 521 | AAAAAATTAAAAAAAGAGGG | GGG |
| 31 | 536 | GAGGGGGGAAATTTTGCCAT | AGG |
| 32 | 537 | AGGGGGGAAATTTTGCCATA | GGG |
| 33 | 541 | TATTAAGGAAAGAAACCCTA | TGG |
| 34 | 556 | TCACATAATAACTCTTATTA | AGG |
| 35 | 620 | TATTATTTTTAATTATTTGT | TGG |
| 36 | 637 | TGTAATAAAAACTAAAGAAA | AGG |
| 37 | 743 | CACTTGGCCCAGGAACATCT | GGG |
| 38 | 744 | TCACTTGGCCCAGGAACATC | TGG |
| 39 | 746 | ATGACTGACCCAGATGTTCC | TGG |
| 40 | 747 | TGACTGACCCAGATGTTCCT | GGG |
| 41 | 753 | AAATAAGGGTCACTTGGCCC | AGG |
| 42 | 759 | TCCCTTAAATAAGGGTCACT | TGG |
| 43 | 767 | GTAAATGCTCCCTTAAATAA | GGG |
| 44 | 768 | GGCCAAGTGACCCTTATTTA | AGG |
| 45 | 768 | TGTAAATGCTCCCTTAAATA | AGG |
| 46 | 769 | GCCAAGTGACCCTTATTTAA | GGG |
| 47 | 783 | ATTTAAGGGAGCATTTACAC | TGG |
| 48 | 2246 | AGGATCGTGACAGATATTCC | TGG |
| 49 | 2253 | AATGTGGCGTCGGTTGTACC | AGG |
| 50 | 2263 | AAAGTTACCGAATGTGGCGT | CGG |
| 51 | 2267 | GGTACAACCGACGCCACATT | CGG |
| 52 | 2269 | AAATTAAAAGTTACCGAATG | TGG |
| 53 | 2296 | ATTGTTGTCTTTTCAAGTTT | AGG |
| 54 | 2319 | AAGACAACAATAATAATAAA | AGG |
| 55 | 2354 | AATTAACTAAGAGAGATTAA | TGG |
| 56 | 2355 | ATTAACTAAGAGAGATTAAT | GGG |
| 57 | 2398 | AATTTGTGATAGGTTAGATG | TGG |
| 58 | 2408 | AGATATGTTTAATTTGTGAT | AGG |
| 59 | 2425 | ACAAATTAAACATATCTTAA | CGG |
| 60 | 2474 | ATATTTTTGAATGAAATCAC | AGG |
| 61 | 2478 | TTTTGAATGAAATCACAGGT | AGG |
| 62 | 2479 | TTTGAATGAAATCACAGGTA | GGG |
| 63 | 2482 | GAATGAAATCACAGGTAGGG | AGG |
| 64 | 2511 | TGGATTCCTATGTTTGGCTT | TGG |
| 65 | 2516 | GAGACTCCAAAGCCAAACAT | AGG |
| 66 | 2517 | AACCTGTGGATTCCTATGTT | TGG |
| 67 | 2526 | AGCCAAACATAGGAATCCAC | AGG |
| 68 | 2531 | ATAGAACAAAAACAAACCTG | TGG |
| 69 | 2566 | CAAACAGAAGAGAAGACAAT | CGG |
| 70 | 2583 | AAACGCTCCCTTGAAGAGGG | TGG |
| 71 | 2586 | CGGTGAATCCACCCTCTTCA | AGG |
| 72 | 2586 | TTGAAACGCTCCCTTGAAGA | GGG |
| 73 | 2587 | GGTGAATCCACCCTCTTCAA | GGG |
| 74 | 2587 | GTTGAAACGCTCCCTTGAAG | AGG |
| 75 | 2611 | GTTGTCGACCGCGAATGCTC | GGG |
| 76 | 2612 | CGTTGTCGACCGCGAATGCT | CGG |
| 77 | 2614 | TTTCAACACCCGAGCATTCG | CGG |
| 78 | 2629 | ATTCGCGGTCGACAACGACC | TGG |
| 79 | 2630 | TTCGCGGTCGACAACGACCT | GGG |
| 80 | 2636 | CGGCAGCGACGGGGAGACCC | AGG |
| 81 | 2645 | TGAAGTAGACGGCAGCGACG | GGG |
| 82 | 2646 | TTGAAGTAGACGGCAGCGAC | GGG |
| 83 | 2647 | ATTGAAGTAGACGGCAGCGA | CGG |
| 84 | 2656 | TCTTTGGGCATTGAAGTAGA | CGG |
| 85 | 2671 | TCTTGCAGCAGTTTCTCTTT | GGG |
| 86 | 2672 | TTCTTGCAGCAGTTTCTCTT | TGG |
| 87 | 2685 | AAAGAGAAACTGCTGCAAGA | AGG |
| 88 | 2704 | AAGGCGTTAAAACCGTCGTC | TGG |

TABLE 2
gRNA and PAM sequences targeted for CuSP-2

| | Position in | | |
|---|---|---|---|
| | SEQ ID | | |
| SEQ ID NO | NO: 89 | Sequence | PAM |
| 92 | 51 | GAAAGAAAAAGAAAGAAAAA | AGG |
| 93 | 64 | AGAAAAAAGGATTGAAATTA | TGG |
| 94 | 89 | ATTAGATCAAAAGTAAGATC | AGG |
| 95 | 114 | ATTACTCTTCCAAGAACAAG | AGG |
| 96 | 116 | CTGCAGAATCCTCTTGTTCT | TGG |
| 97 | 128 | CTTGTTCTTGGAAGAGTAAT | TGG |
| 98 | 153 | ATTTTAATGGTTGGACTGAA | GGG |
| 99 | 154 | CATTTTAATGGTTGGACTGA | AGG |
| 100 | 162 | GTGACAGACATTTTAATGGT | TGG |
| 101 | 166 | GAAAGTGACAGACATTTTAA | TGG |
| 102 | 190 | ATTGAGGACTTGTTTGTTAT | TGG |
| 103 | 203 | AATAACAAACAAGTCCTCAA | TGG |
| 104 | 206 | GGAAGAATTCATGGCCATTG | AGG |
| 105 | 215 | GAGAAGAAGGGAAGAATTCA | TGG |
| 106 | 227 | GTTTGAAGGAAAGAGAAGAA | GGG |
| 107 | 228 | GGTTTGAAGGAAAGAGAAGA | AGG |
| 108 | 241 | AATATGAACCCTAGGTTTGA | AGG |
| 109 | 243 | CTTCTCTTTCCTTCAAACCT | AGG |
| 110 | 244 | TTCTCTTTCCTTCAAACCTA | GGG |
| ill | 249 | TCTCCTTGAATATGAACCCT | AGG |
| 112 | 257 | AAACCTAGGGTTCATATTCA | AGG |
| 113 | 286 | TATGAGATCATTGTTCACTC | TGG |
| 114 | 318 | CTTTTATTTAAGAAAAAAAA | AGG |
| 115 | 358 | AGCTTTTTGTTTTTTTTTTT | TGG |
| 116 | 369 | TTTTTTTTTTGGTTAGGTTA | TGG |
| 117 | 385 | CACTAGGGCCAGGAACATCA | GGG |
| 118 | 386 | TCACTAGGGCCAGGAACATC | AGG |
| 119 | 388 | ATGGTTGACCCTGATGTTCC | TGG |
| 120 | 395 | AAGTAAGGATCACTAGGGCC | AGG |
| 121 | 400 | CCCTCAAGTAAGGATCACTA | GGG |
| 122 | 401 | TCCCTCAAGTAAGGATCACT | AGG |
| 123 | 410 | GCCCTAGTGATCCTTACTTG | AGG |
| 124 | 410 | TGAAGGTGTTCCCTCAAGTA | AGG |
| 125 | 411 | CCCTAGTGATCCTTACTTGA | GGG |
| 126 | 425 | ACTTGAGGGAACACCTTCAC | TGG |
| 127 | 427 | TTTTATATATATACCAGTGA | AGG |
| 128 | 455 | GCTCAAAATTTAAAAATGAA | TGG |
| 129 | 516 | GAAATTACTATTTTGGAAAT | GGG |
| 130 | 517 | AGAAATTACTATTTTGGAAA | TGG |
| 131 | 523 | ATAGAAAGAAATTACTATTT | TGG |
| 132 | 552 | TTGAGAAAATCATACAAAAT | AGG |
| 133 | 566 | ATTTTGTATGATTTTCTCAA | TGG |
| 134 | 659 | AAATTTATTGTTTTGGTAAT | GGG |
| 135 | 660 | TAAATTTATTGTTTTGGTAA | TGG |
| 136 | 666 | CAAACATAAATTTATTGTTT | TGG |
| 137 | 692 | TTTGTTGATAAAATTGAAAT | TGG |
| 138 | 707 | TTTCAATTTTATCAACAAAT | TGG |
| 139 | 713 | TTTTATCAACAAATTGGAAA | TGG |
| 140 | 731 | CCAAATAAAGGTAAAAAGTT | AGG |
| 141 | 742 | CCTAACTTTTTACCTTTATT | TGG |
| 142 | 743 | AAATAAAAAGATCCAAATAA | AGG |
| 143 | 760 | TTTGGATCTTTTTATTTTTC | AGG |
| 144 | 764 | GATCTTTTTATTTTTCAGGT | TGG |
| 145 | 780 | AGGTTGGTGACTGACATTCC | AGG |
| 146 | 787 | AAAGTAGCATCAGTAGTTCC | TGG |
| 147 | 801 | GGAACTACTGATGCTACTTT | TGG |
| 148 | 806 | TACTGATGCTACTTTTGGTA | AGG |
| 149 | 876 | TAATTTTAATTGAAAATGTT | TGG |
| 150 | 877 | AATTTTAATTGAAAATGTTT | GGG |
| 151 | 883 | AATTGAAAATGTTTGGGATG | TGG |
| 152 | 902 | GTGGAATATATATATGAGAC | AGG |
| 153 | 939 | TGAATTCCTATTGTTGGCTT | TGG |
| 154 | 944 | GAAATTCCAAAGCCAACAAT | AGG |
| 155 | 945 | AACCTGTGAATTCCTATTGT | TGG |
| 156 | 954 | AGCCAACAATAGGAATTCAC | AGG |
| 157 | 990 | TGTTCAAACAAAAACAGCGT | CGG |
| 158 | 1014 | AAACGATCCCTTGATGAAGG | AGG |
| 159 | 1017 | TAGTGAATCCTCCTTCATCA | AGG |
| 160 | 1017 | TTGAAACGATCCCTTGATGA | AGG |
| 161 | 1018 | AGTGAATCCTCCTTCATCAA | GGG |
| 162 | 1060 | ATTTTCTTGTGAGAATGATT | TGG |
| 163 | 1061 | TTTTCTTGTGAGAATGATTT | GGG |
| 164 | 1077 | TTGAAATAGACAGCAGCAAC | AGG |
| 165 | 1113 | CTCAAAGAGAAACTGCTGCA | AGG |
| 166 | 1116 | AAAGAGAAACTGCTGCAAGG | AGG |

TABLE 3
gRNA and PAM sequences targeted for CuSP-3

| | Position in | | |
|---|---|---|---|
| SEQ ID | SEQ ID | | |
| NO | NO: 167 | Sequence | PAM |
| 170 | 45 | TTCAAGATAGTATTTCAAAA | GGG |
| 171 | 46 | GTTCAAGATAGTATTTCAAA | AGG |
| 172 | 68 | AACTAAAAGAAATAAAAAAA | AGG |
| 173 | 86 | TTTATTTCTTTTAGTTTCTA | TGG |
| 174 | 92 | TCTTTTAGTTTCTATGGCGA | TGG |
| 175 | 93 | CTTTTAGTTTCTATGGCGAT | GGG |
| 176 | 94 | TTTTAGTTTCTATGGCGATG | GGG |
| 177 | 112 | CCAACCACGAGAGGATCGGA | CGG |
| 178 | 116 | TCCTCCAACCACGAGAGGAT | CGG |
| 179 | 119 | GATGCCGTCCGATCCTCTCG | TGG |
| 180 | 121 | ACTACTCCTCCAACCACGAG | AGG |
| 181 | 123 | CCGTCCGATCCTCTCGTGGT | TGG |
| 182 | 126 | TCCGATCCTCTCGTGGTTGG | AGG |
| 183 | 135 | CTCGTGGTTGGAGGAGTAGT | CGG |
| 184 | 164 | TGTCGATGCAATTTCTCCTA | CGG |
| 185 | 169 | GTGACGGTCATCTTAACCGT | AGG |
| 186 | 185 | CTTGTTTGAATGGTAAGTGA | CGG |
| 187 | 195 | TGCACACCTTCTTGTTTGAA | TGG |
| 188 | 200 | CACTTACCATTCAAACAAGA | AGG |
| 189 | 210 | TCAAACAAGAAGGTGTGCAA | TGG |
| 190 | 211 | CAAACAAGAAGGTGTGCAAT | GGG |
| 191 | 235 | GGTTTTAGGGTTACAAAATT | TGG |
| 192 | 248 | AACCTCAACCTTAGGTTTTA | GGG |
| 193 | 249 | GAACCTCAACCTTAGGTTTT | AGG |
| 194 | 251 | TTTTGTAACCCTAAAACCTA | AGG |
| 195 | 256 | CCTCCAAGAACCTCAACCTT | AGG |
| 196 | 257 | AACCCTAAAACCTAAGGTTG | AGG |
| 197 | 264 | AAACCTAAGGTTGAGGTTCT | TGG |
| 198 | 267 | CCTAAGGTTGAGGTTCTTGG | AGG |
| 199 | 282 | CCAGTGTGAAGAATGATCTA | AGG |
| 200 | 293 | CCTTAGATCATTCTTCACAC | TGG |
| 201 | 329 | AAGAAAAGAAAAGAAAAGAT | GGG |
| 202 | 330 | AAAGAAAAGAAAAGAAAAGA | TGG |
| 203 | 383 | GAAACTATATAAATTAAGTA | CGG |
| 204 | 415 | TCAACTAATTATTTTTTTCC | AGG |
| 205 | 422 | CATCTGGATCAGTCATGACC | TGG |
| 206 | 438 | TCACTTGGACCAGGAACATC | TGG |
| 207 | 440 | ATGACTGATCCAGATGTTCC | TGG |
| 208 | 447 | AAGTAAGGATCACTTGGACC | AGG |
| 209 | 453 | TCTCTCAAGTAAGGATCACT | TGG |
| 210 | 462 | TGGAGGTGTTCTCTCAAGTA | AGG |
| 211 | 477 | ACTTGAGAGAACACCTCCAC | TGG |
| 212 | 479 | TTAGAATATGATACCAGTGG | AGG |
| 213 | 482 | GAATTAGAATATGATACCAG | TGG |
| 214 | 510 | TTTTTACACCCTTACAGATG | GGG |
| 215 | 511 | ATTTTTACACCCTTACAGAT | GGG |
| 216 | 512 | ATTCAAGAACCCCATCTGTA | AGG |
| 217 | 512 | CATTTTTACACCCTTACAGA | TGG |
| 218 | 513 | TTCAAGAACCCCATCTGTAA | GGG |
| 219 | 614 | AGAATATGTCAAGTATTGTT | TGG |
| 220 | 615 | GAATATGTCAAGTATTGTTT | GGG |
| 221 | 621 | GTCAAGTATTGTTTGGGAAT | TGG |
| 222 | 686 | GTTCTAAAAATCAAAATACA | AGG |
| 223 | 687 | TTCTAAAAATCAAAATACAA | GGG |
| 224 | 694 | AATCAAAATACAAGGGTTTT | AGG |
| 225 | 735 | TACGAACACTAAAATTTTAA | AGG |
| 226 | 736 | ACGAACACTAAAATTTTAAA | GGG |
| 227 | 793 | AAAGAGAAATCATTCTTTAT | TGG |
| 228 | 818 | ACATGTGTTAGTTAAGTCGA | AGG |
| 229 | 892 | TAGTTATACAATTGAAATCG | AGG |
| 230 | 917 | GTATAACTACTGAAATAATT | TGG |
| 231 | 943 | ATATATAACTTTGATATTTC | AGG |
| 232 | 963 | AGGATAGTTACAGACATTCC | AGG |
| 233 | 964 | GGATAGTTACAGACATTCCA | GGG |
| 234 | 970 | AAAGTGGCATCCGTCGTCCC | TGG |
| 235 | 971 | TACAGACATTCCAGGGACGA | CGG |
| 236 | 984 | GGGACGACGGATGCCACTTT | TGG |
| 237 | 986 | AAATGAAGTTTTACCAAAAG | TGG |
| 238 | 1056 | ATATACATACTAGATACTGA | TGG |
| 239 | 1057 | TATACATACTAGATACTGAT | GGG |
| 240 | 1076 | TGGGAAAATTAATAAAATAT | AGG |
| 241 | 1113 | TGTATCCCTATGTTTGGACT | TGG |
| 242 | 1118 | GAAGAACCAAGTCCAAACAT | AGG |
| 243 | 1119 | AAGAACCAAGTCCAAACATA | GGG |
| 244 | 1119 | TATCTGTGTATCCCTATGTT | TGG |
| 245 | 1148 | TTCTTTTTTGCTTGTACAAT | AGG |
| 246 | 1185 | TCTCTTGAAGGGTGTGGTGG | CGG |
| 247 | 1188 | CCGTCTCTTGAAGGGTGTGG | TGG |
| 248 | 1191 | AAACCGTCTCTTGAAGGGTG | TGG |
| 249 | 1196 | AATTAAAACCGTCTCTTGAA | GGG |
| 250 | 1197 | GAATTAAAACCGTCTCTTGA | AGG |
| 251 | 1199 | CCACCACACCCTTCAAGAGA | CGG |
| 252 | 1254 | ATGAAATAGACAGCAGCAAC | AGG |
| 253 | 1275 | CTGTCTATTTCATTGCTCAA | AGG |
| 254 | 1287 | TATCTTCTTCTGGCAGCAGT | AGG |
| 255 | 1297 | GTGTGTGTGTTATCTTCTTC | TGG |

Reference is made to Table 4 presenting a summary of the sequences within the scope of the current invention.

TABLE 4
Summary of sequences within the scope of the present invention

| Sequence | | | |
|---|---|---|---|
| type | CuSP-1 | CuSP-2 | CuSP-3 |
| Genomic | SEQ ID NO: 1 | SEQ ID NO: 89 | SEQ ID NO: 167 |
| sequence | | | |
| Coding | SEQ ID NO: 2 | SEQ ID NO: 90 | SEQ ID NO: 168 |
| sequence | | | |
| (CDS) | | | |
| Amino acid | SEQ ID NO: 3 | SEQ ID NO: 91 | SEQ ID NO: 169 |
| sequence | | | |
| gRNA | SEQ ID NO: 4- | SEQ ID NO: 92- | SEQ ID NO: 170- |
| sequence | SEQ ID NO: 88 | SEQ ID NO: 166 | SEQ ID NO: 255 |

The above gRNA molecules have been cloned into suitable vectors and their sequence has been verified. In addition different Cas9 versions have been analyzed for optimal compatibility between the Cas9 protein activity and the gRNA molecule in the Cucumber plant.

The efficiency of the designed gRNA molecules have been validated by transiently transforming Cucumber tissue culture. A plasmid carrying gRNA sequence together with the Cas9 gene has been transformed into Cucumber protoplasts. The protoplast cells have been grown for a short period of time and then were analyzed for existence of genome editing events. The positive constructs have been subjected to the herein established stable transformation protocol into Cucumber plant tissue for producing genome edited Cucumber plants in SP genes.

Stage 3: Transforming Cucumber plants using Agrobacterium or biolistics (gene gun) methods. For Agrobacterium and bioloistics, a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various Cucumber tissues was performed using particle bombardment of:
DNA vectors
Ribonucleoprotein complex (RNP's)

According to further embodiments of the present invention, transformation of various Cucumber tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

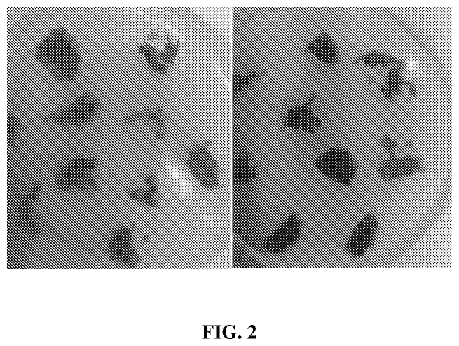

Reference is now made to FIG. 2 photographically presenting regenerated transformed Cucumber tissue. Cucumber seeds were germinated in the dark for 3 days after which cotyledons were excised and placed on regeneration medium. Two to three weeks after excision, regenerated cucumber seedlings began to emerge at the cotyledon cut site (marked with red *).

According to further embodiments of the present invention, additional transformation tools were used in Cucumber, including, but not limited to:
Protoplast PEG transformation
Extend RNP use
Directed editing screening using fluorescent tags
Electroporation

Stage 4: Regeneration in tissue-culture. When transforming DNA constructs into the plant, antibiotics is used for selection of positive transformed plants. An improved regeneration protocol was herein established for the Cucumber plant.

Stage 5: Selection of positive transformants. Once regenerated plants appear in tissue culture, DNA is extracted from leaf sample of the transformed plant and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

Analysis of CRISPR/Cas9 cleavage activity was performed by digestion of the resulted PCR amplicon containing the gene specific gRNA sequence, by RNP complex containing Cas9. The analysis included the following steps:
1) Amplicon was isolated from two exemplified Cucumber strains by primers flanking the sequence of the gene of interest targeted by the predesigned sgRNA.
2) RNP complex was incubated with the isolated amplicon.
3) The reaction mix was then loaded on agarose gel to evaluate Cas9 cleavage activity at the target site.

Stage 6: Selection of transformed Cucumber plants with gene edited versions of the targeted genes CuSP-1, CuSP-1 and/or CuSP-1. These plants were further examined for reduced expression (at the transcription and/or post transcription levels) of at least one of these genes. In addition, transformed Cucumber plants presenting sp related phenotypes as described above, were selected. It is within the scope that different gRNA promoters were tested in order to maximize editing efficiency.

### REFERENCES

Li Tingdong, Yang Xinping, Yu Yuan, Si Xiaomin, Zhai Xiawan, Zhang Huawei, Dong Wenxia, Gao Caixia, and Xu Cao. “Domestication of wild tomato is accelerated by genome editing”. Nature biotechnology, (2018). doi:10.1038/nbt.4273.
Zsögön Agustin, Čerma\acute{k} Tomás, Rezende Naves Emmanuel, Morato Notini Marcela, Edel Kai H, Weinl Stefan, Freschi Luciano, Voytas Daniel F, Kudla Jörg, and Eustáquio Pereira Peres Lázaro. “De novo domestication of wild tomato using genome editing”. Nature Biotechnology, (2018). doi: 10.1038/nbt.4272.
Lemmon Zachary H, Reem Nathan T., Dalrymple Justin, Soyk Sebastian, Swartwood Kerry E., Rodriguez-Leal Daniel, Van Eck Joyce, and Lippman Zachary B. “Rapid improvement of domestication traits in an orphan crop by genome editing”. Nature Plants 4 (2018): 766-770.
Xie Kabin, and Yinong Yang. “RNA-guided genome editing in plants using a CRISPR-Cas system”. Molecular plant 6 (2013): 1975-1983.

1-80. (canceled)

81. A modified Cucumber plant exhibiting at least one improved domestication trait, wherein said modified plant comprises at least one targeted genome editing modification in at least one Cucumber SELF PRUNING (SP) (CuSP) gene encoding a sequence selected from a sequence comprising at least 80% identity to SEQ ID NO:3 or to SEQ ID NO:169, and a sequence comprising at least 99.45% identity to SEQ ID NO:91, said at least one modification confers reduced expression of said at least one CuSP gene.

82. The modified Cucumber plant according to claim 81, wherein said modified cucumber plant exhibits at least one improved domestication trait as compared to a corresponding Cucumber plant lacking said genetic modification and/or said plant has decreased expression levels of at least one of said CuSP genes.

83. The modified Cucumber plant according to claim 81, wherein said modified cucumber plant comprises at least one genetic modification introduced in said at least one CuSP gene using targeted genome modification.

84. The modified Cucumber plant according to claim 83, wherein said genetic modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

85. The modified Cucumber plant according to claim 81, wherein said genetic modification is at least one of the following:
a. a missense mutation, nonsense mutation, insertion, deletion, indel, substitution or duplication;
b. in the coding region of said gene, a mutation in the regulatory region of said gene, or an epigenetic factor;
c. a silencing mutation, a knockdown mutation, a knockout mutation, a loss of function mutation or any combination thereof; and
d. generated in planta.

86. The modified Cucumber plant according to claim 81 wherein said genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO:4-88, 92-166, 170-255 and any combination thereof.

87. The modified Cucumber plant according to claim 81, wherein said plant is at least one of the following:
a. semi-determinant or has determinant growth habit;
b. flowers earlier than a corresponding Cucumber plant lacking said genetic modification;
c. exhibits improved earliness as compared to a corresponding Cucumber plant lacking said genetic modification;
d. exhibits suppressed sympodial shoot termination as compared to a corresponding Cucumber plant lacking said genetic modification; and
e. exhibits similar sympodial shoot termination as compared to a corresponding Cucumber plant lacking said genetic modification.

88. The modified Cucumber plant according to claim 81, wherein said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

89. A plant part, plant cell, plant fruit, plant seed or tissue culture of regenerable cells, protoplasts or callus of a modified cucumber plant according to claim 81, wherein said plant part, plant cell, plant fruit or plant seed comprises at least one genetic modification conferring reduced expression of the at least one Cucumber SELF PRUNING (SP) (CuSP) gene.

90. A method for producing a modified Cucumber plant exhibiting at least one improved domestication trait, wherein said method comprises steps of genetically modifying by genome editing at least one Cucumber SELF PRUNING (SP) (CuSP) gene, said gene encoding a sequence selected from a sequence comprising at least 80% identity to SEQ ID NO:3 or to SEQ ID NO:169, and a sequence comprising at least 99.45% identity to SEQ ID NO:91.

91. The method according to claim 90, comprises steps of producing the modified Cucumber plant using targeted genome modification, by genetically introducing a loss of function mutation in said at least one Cucumber SELF PRUNING (SP) (CuSP) gene.

92. The method according to claim 90, wherein at least one of the following holds true:
a. said genetic modification confers reduced expression of the at least one Cucumber SELF PRUNING (SP) (CuSP) gene; and
b. said modified Cucumber plant exhibits at least one improved domestication trait as compared to a corresponding Cucumber plant lacking said genetic modification.

93. The method according to claim 90, wherein said method further comprises steps of:
a. identifying at least one Cucumber SP (CuSP) gene or allele;
b. synthetizing at least one guide RNA (gRNA) comprising a nucleotide sequence complementary to said at least one identified CuSP allele;
c. transforming Cucumber plant cells with a construct comprising (a) Cas nucleotide sequence operably linked to said at least one gRNA, or (b) a ribonucleoprotein (RNP) complex comprising Cas protein and said at least one gRNA;
d. screening the genome of said transformed plant cells for induced targeted loss of function mutation in at least one of said CuSP allele or gene;
e. regenerating Cucumber plant carrying said loss of function mutation in at least one of said CuSP allele or gene; and
f. screening said regenerated plants for a Cucumber plant with improved domestication trait.

94. The method according to claim 90, wherein said genetic modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

95. The method according to claim 90, wherein said genetic modification is generated in planta via introduction of a construct comprising (a) Cas DNA and gRNA sequence selected from the group consisting of SEQ ID NO:4-88, 92-166, 170-255 and any combination thereof, or (b) a ribonucleoprotein (RNP) complex comprising Cas protein and gRNA sequence selected from the group consisting of SEQ ID NO: 4-88, 92-166, 170-255 and any combination thereof.

96. The method according to claim 90, wherein said modified plant is at least one of:
a. has decreased expression levels of at least one of said CuSP genes;
b. semi-determinant;
c. has determinant growth habit;
d. flowers earlier than a corresponding Cucumber plant lacking said genetic modification
e. exhibits improved earliness as compared to a corresponding Cucumber plant lacking said genetic modification
f. exhibits suppressed sympodial shoot termination as compared to a corresponding Cucumber plant lacking said genetic modification;
g. exhibits similar sympodial shoot termination as compared to a corresponding Cucumber plant lacking said genetic modification; and
h. exhibits suppressed or reduced day-length sensitivity as compared to a corresponding Cucumber plant lacking said genetic modification.

97. A modified Cucumber plant, plant part, plant fruit, plant cell, plant seed or tissue culture of regenerable cells, protoplasts or callus produced or obtained by the method according to claim 90.

98. The method according to claim 90, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

99. An isolated nucleotide sequence selected from at least one of:
a. a nucleotide sequence encoding a sequence selected from a sequence comprising at least 80% identity to SEQ ID NO:3 or to SEQ ID NO:169, and a sequence comprising at least 99.45% identity to SEQ ID NO:91; and
b. a nucleotide sequence having at least 75% sequence identity to a CuSP-targeted gRNA nucleotide sequence as set forth in SEQ ID NO:4-88, 92-166 and 170-255.

100. A method of utilizing the nucleotide sequence according to claim 99 as following:
a. using the nucleotide sequence as set forth in at least one of SEQ ID NO:4-88 and any combination thereof for targeted genome modification of Cucumber SP-1 (CuSP-1) allele or gene;
b. using the nucleotide sequence as set forth in at least one of SEQ ID NO:92-166 and any combination thereof for targeted genome modification of Cucumber SP-2 (CuSP-2) allele or gene; and
c. using the nucleotide sequence as set forth in at least one of SEQ ID NO:170-255 and any combination thereof for targeted genome modification of Cucumber SP-3 (CuSP-3) allele or gene.


SEQUENCE LISTING (255 sequences)

SEQ ID NO: 1 - DNA, 3110 bp, Cucumis sativus
gcaaaaaatagggtttgaaagaaagaaaaaaaaaaaacttttttctttttctaaatctgagtgctttttgagaaatggcaagatcatcagcaatgtcctcagaacctcttgttgttggtagagtgattggtgatgttcttgattccttcacacaaagcatgaagatgagtgttttttatagcaataataagcaagttttcaatggccatgagttctttccttctgctgttgctgctaaacctagggctgaaatccatggtggtgacctgagatctttcttcactctggtttgtaattcttttaacttcatttccttgtttgtttatctcaaacttattacaaaaaatgatttatctgaggcttggatctgatcttattctgcatgttttgagttgtgtgtggtctttaaattcgaagatatatagttttgttgcttaaaatattgattatatcgttcataggtttaaatttctactcttgcatgttgaataaatttaaaaagaaaaaaattaaaaaaagaggggggaaattttgccatagggtttctttccttaataagagttattatgtgatttaacttaatatttgacacttttaaataatattatttttaattatttgttggaattaccttttctttagtttttattacatttttaattattatatatatctcttaatactttgattatttgatttaattagttttttctttttgttttcagattatgactgacccagatgttcctgggccaagtgacccttatttaagggagcatttacactggtaactaaacatttacatatatatgtttatgatatttcttttattatttttttccttctaaggctatatatatatacatatatttatttacatgacttcataaaaccaaccaaattaatttgacctttgttcgagtacaacataaagtactggaatttgaacctggaacatccaatcaaatttgaatttttgaatttgaggaagaataatgcaaaaatcatcgtcgttactctattttggattttggctaattttgcatatttttcttttattttcattttgcctacaacattttttttttgttctgatttgccttttatttggcgttatacatttttaggctttttttttttaaatttttggaagaaaaatttgaaaataaataatctaaggatttgcctcttacatttaataaaataaaataaaattataaagagaaaaaaaatacaagtattatttaaatagttgtttacaagtattatttaataatctatagtatttatttagggtttttttcaaaagtagaagaaaaaaaaaacaaatatttactttactttgtataattattcctgcattcattattaagggcatgataatcactggattggactccttatttgatatctcttaagttcctaacttagggtaaatatttgtcaacacttttagcaattttcttttttaagttaaacaaaggtgtggggactaaactttcaattttagcatttttgctaataataataataatctttttatccatgcatatatgtgcaacctttcgacaagaatttttaattcaaaattatcaatgaaatcaatgagttactttcactaaaatagtttattttataatatgtgggtaccagaatatcaaacatgaatgataatatcgactaatttaaatatgttaaaatgtcaagaatagtatttctataaaaaaaaaattaaaaagtagatgggtttgaataaaaatatattttaatttgcaataacaaacgagttaatatgtgtaatatttatattagtgatatgcatggttacttcataaaatgtagacctacttctttatgtcaaatgtggggcacatgagcgaaaaaaccgctcggccttacacgttcgaaccattcacacaccttctatttttgcatctcaattcattgtatcaaaaccatcaaatcttcaccttatgcacattttttcattacaacgatagattaccaaatagagatgtccattttttccgcaagaatattagaagtcatgcatccatttttttctccgctcgttcattaacgtctctaacaccaaatcttctctcccaaacacttttggagaaaaaaaagttgttttcattttttttttatttaacttctgcaatcgataacatatgtaaaacttgttagaaaagataagtggttttgactatggaatctctaattgcaggatcgtgacagatattcctggtacaaccgacgccacattcggtaacttttaatttcttgcctaaacttgaaaagacaacaataataataaaaggttttaaacttgtaattaactaagagagattaatgggacaattgtgtgaattgaaatttgaaatttctccacatctaacctatcacaaattaaacatatcttaacggttataaactactaaatattgaaagaaatatttttgaatgaaatcacaggtagggaggtagtaagctacgagactccaaagccaaacataggaatccacaggtttgtttttgttctattcaaacagaagagaagacaatcggtgaatccaccctcttcaagggagcgtttcaacacccgagcattcgcggtcgacaacgacctgggtctccccgtcgctgccgtctacttcaatgcccaaagagaaactgctgcaagaaggcgttaaaaccgtcgtctggcaacagtagtgatcccagtggaccaaaatattttgaattctagaagaaaattaagaaaactatagtattaatatattaatctgagtctttctatttcctctccctttaacaatattaaccactttgtatggtgatgatagaaggaaaagggagggagattagagataattctcagatttgtatattagataaaaataatctatatttgcatgaagggagaataatttggacatgcaaaaaggaagaaacgtcgccgttttcttttcctctttggtgtctttaattagtttttattgtatgtgctaatgaatttagtttgagatgatttacaattattaccacagtttgtttttagtttcaaattaaaatatatatgaaatggtccatttggtaacttcttt

SEQ ID NO: 2 - DNA, 534 bp, Cucumis sativus
atggcaagatcatcagcaatgtcctcagaacctcttgttgttggtagagtgattggtgatgttcttgattccttcacacaaagcatgaagatgagtgttttttatagcaataataagcaagttttcaatggccatgagttctttccttctgctgttgctgctaaacctagggctgaaatccatggtggtgacctgagatctttcttcactctgattatgactgacccagatgttcctgggccaagtgacccttatttaagggagcatttacactggatcgtgacagatattcctggtacaaccgacgccacattcggtagggaggtagtaagctacgagactccaaagccaaacataggaatccacaggtttgtttttgttctattcaaacagaagagaagacaatcggtgaatccaccctcttcaagggagcgtttcaacacccgagcattcgcggtcgacaacgacctgggtctccccgtcgctgccgtctacttcaatgcccaaagagaaactgctgcaagaaggcgttaa

SEQ ID NO: 3 - protein, 177 aa, Cucumis sativus
MARSSAMSSEPLVVGRVIGDVLDSFTQSMKMSVFYSNNKQVFNGHEFFPSAVAAKPRAEIHGGDLRSFFTLIMTDPDVPGPSDPYLREHLHWIVTDIPGTTDATFGREVVSYETPKPNIGIHRFVFVLFKQKRRQSVNPPSSRERFNTRAFAVDNDLGLPVAAVYFNAQRETAARRR

SEQ ID NO: 4 - DNA, 20 bp, Cucumis_sativus
tctgagtgctttttgagaaa

SEQ ID NO: 5 - DNA, 20 bp, Cucumis_sativus
accaacaacaagaggttctg

SEQ ID NO: 6 - DNA, 20 bp, Cucumis_sativus
atcactctaccaacaacaag

SEQ ID NO: 7 - DNA, 20 bp, Cucumis_sativus
tcctcagaacctcttgttgt

SEQ ID NO: 8 - DNA, 20 bp, Cucumis_sativus
cttgttgttggtagagtgat

SEQ ID NO: 9 - DNA, 20 bp, Cucumis_sativus
catcttcatgctttgtgtga

SEQ ID NO: 10 - DNA, 20 bp, Cucumis_sativus
aataataagcaagttttcaa

SEQ ID NO: 11 - DNA, 20 bp, Cucumis_sativus
cagcagaaggaaagaactca

SEQ ID NO: 12 - DNA, 20 bp, Cucumis_sativus
ggtttagcagcaacagcaga

SEQ ID NO: 13 - DNA, 20 bp, Cucumis_sativus
ctgctgttgctgctaaacct

SEQ ID NO: 14 - DNA, 20 bp, Cucumis_sativus
tgctgttgctgctaaaccta

SEQ ID NO: 15 - DNA, 20 bp, Cucumis_sativus
ccaccatggatttcagccct

SEQ ID NO: 16 - DNA, 20 bp, Cucumis_sativus
aaacctagggctgaaatcca

SEQ ID NO: 17 - DNA, 20 bp, Cucumis_sativus
cctagggctgaaatccatgg

SEQ ID NO: 18 - DNA, 20 bp, Cucumis_sativus
aagatctcaggtcaccacca

SEQ ID NO: 19 - DNA, 20 bp, Cucumis_sativus
ccagagtgaagaaagatctc

SEQ ID NO: 20 - DNA, 20 bp, Cucumis_sativus
cctgagatctttcttcactc

SEQ ID NO: 21 - DNA, 20 bp, Cucumis_sativus
aagtttgagataaacaaaca

SEQ ID NO: 22 - DNA, 20 bp, Cucumis_sativus
tacaaaaaatgatttatctg

SEQ ID NO: 23 - DNA, 20 bp, Cucumis_sativus
aaaatgatttatctgaggct

SEQ ID NO: 24 - DNA, 20 bp, Cucumis_sativus
tgcatgttttgagttgtgtg

SEQ ID NO: 25 - DNA, 20 bp, Cucumis_sativus
atattgattatatcgttcat

SEQ ID NO: 26 - DNA, 20 bp, Cucumis_sativus
aagaaaaaaattaaaaaaag

SEQ ID NO: 27 - DNA, 20 bp, Cucumis_sativus
agaaaaaaattaaaaaaaga

SEQ ID NO: 28 - DNA, 20 bp, Cucumis_sativus
gaaaaaaattaaaaaaagag

SEQ ID NO: 29 - DNA, 20 bp, Cucumis_sativus
aaaaaaattaaaaaaagagg

SEQ ID NO: 30 - DNA, 20 bp, Cucumis_sativus
aaaaaattaaaaaaagaggg

SEQ ID NO: 31 - DNA, 20 bp, Cucumis_sativus
gaggggggaaattttgccat

SEQ ID NO: 32 - DNA, 20 bp, Cucumis_sativus
aggggggaaattttgccata

SEQ ID NO: 33 - DNA, 20 bp, Cucumis_sativus
tattaaggaaagaaacccta

SEQ ID NO: 34 - DNA, 20 bp, Cucumis_sativus
tcacataataactcttatta

SEQ ID NO: 35 - DNA, 20 bp, Cucumis_sativus
tattatttttaattatttgt

SEQ ID NO: 36 - DNA, 20 bp, Cucumis_sativus
tgtaataaaaactaaagaaa

SEQ ID NO: 37 - DNA, 20 bp, Cucumis_sativus
cacttggcccaggaacatct

SEQ ID NO: 38 - DNA, 20 bp, Cucumis_sativus
tcacttggcccaggaacatc

SEQ ID NO: 39 - DNA, 20 bp, Cucumis_sativus
atgactgacccagatgttcc

SEQ ID NO: 40 - DNA, 20 bp, Cucumis_sativus
tgactgacccagatgttcct

SEQ ID NO: 41 - DNA, 20 bp, Cucumis_sativus
aaataagggtcacttggccc

SEQ ID NO: 42 - DNA, 20 bp, Cucumis_sativus
tcccttaaataagggtcact

SEQ ID NO: 43 - DNA, 20 bp, Cucumis_sativus
gtaaatgctcccttaaataa

SEQ ID NO: 44 - DNA, 20 bp, Cucumis_sativus
tgtaaatgctcccttaaata

SEQ ID NO: 45 - DNA, 20 bp, Cucumis_sativus
gccaagtgacccttatttaa

SEQ ID NO: 46 - DNA, 20 bp, Cucumis_sativus
atttaagggagcatttacac

SEQ ID NO: 47 - DNA, 20 bp, Cucumis_sativus
aggatcgtgacagatattcc

SEQ ID NO: 48 - DNA, 20 bp, Cucumis_sativus
aatgtggcgtcggttgtacc

SEQ ID NO: 49 - DNA, 20 bp, Cucumis_sativus
aaagttaccgaatgtggcgt

SEQ ID NO: 50 - DNA, 20 bp, Cucumis_sativus
ggtacaaccgacgccacatt

SEQ ID NO: 51 - DNA, 20 bp, Cucumis_sativus
aaattaaaagttaccgaatg

SEQ ID NO: 52 - DNA, 20 bp, Cucumis_sativus
attgttgtcttttcaagttt

SEQ ID NO: 53 - DNA, 20 bp, Cucumis_sativus
aagacaacaataataataaa

SEQ ID NO: 54 - DNA, 20 bp, Cucumis_sativus
aattaactaagagagattaa

SEQ ID NO: 55 - DNA, 20 bp, Cucumis_sativus
attaactaagagagattaat

SEQ ID NO: 56 - DNA, 20 bp, Cucumis_sativus
aatttgtgataggttagatg

SEQ ID NO: 57 - DNA, 20 bp, Cucumis_sativus
agatatgtttaatttgtgat

SEQ ID NO: 58 - DNA, 20 bp, Cucumis_sativus
acaaattaaacatatcttaa

SEQ ID NO: 59 - DNA, 20 bp, Cucumis_sativus
atatttttgaatgaaatcac

SEQ ID NO: 60 - DNA, 20 bp, Cucumis_sativus
ttttgaatgaaatcacaggt

SEQ ID NO: 61 - DNA, 20 bp, Cucumis_sativus
tttgaatgaaatcacaggta

SEQ ID NO: 62 - DNA, 20 bp, Cucumis_sativus
gaatgaaatcacaggtaggg

SEQ ID NO: 63 - DNA, 20 bp, Cucumis_sativus
tggattcctatgtttggctt

SEQ ID NO: 64 - DNA, 20 bp, Cucumis_sativus
gagactccaaagccaaacat

SEQ ID NO: 65 - DNA, 20 bp, Cucumis_sativus
aacctgtggattcctatgtt

SEQ ID NO: 66 - DNA, 20 bp, Cucumis_sativus
agccaaacataggaatccac

SEQ ID NO: 67 - DNA, 20 bp, Cucumis_sativus
atagaacaaaaacaaacctg

SEQ ID NO: 68 - DNA, 20 bp, Cucumis_sativus
caaacagaagagaagacaat

SEQ ID NO: 69 - DNA, 20 bp, Cucumis_sativus
aaacgctcccttgaagaggg

SEQ ID NO: 70 - DNA, 20 bp, Cucumis_sativus
ttgaaacgctcccttgaaga

SEQ ID NO: 71 - DNA, 20 bp, Cucumis_sativus
gttgaaacgctcccttgaag

SEQ ID NO: 72 - DNA, 20 bp, Cucumis_sativus
gttgtcgaccgcgaatgctc

SEQ ID NO: 73 - DNA, 20 bp, Cucumis_sativus
cgttgtcgaccgcgaatgct

SEQ ID NO: 74 - DNA, 20 bp, Cucumis_sativus
tttcaacacccgagcattcg

SEQ ID NO: 75 - DNA, 20 bp, Cucumis_sativus
attcgcggtcgacaacgacc

SEQ ID NO: 76 - DNA, 20 bp, Cucumis_sativus
ttcgcggtcgacaacgacct

SEQ ID NO: 77 - DNA, 20 bp, Cucumis_sativus
cggcagcgacggggagaccc

SEQ ID NO: 78 - DNA, 20 bp, Cucumis_sativus
tgaagtagacggcagcgacg

SEQ ID NO: 79 - DNA, 20 bp, Cucumis_sativus
ttgaagtagacggcagcgac

SEQ ID NO: 80 - DNA, 20 bp, Cucumis_sativus
attgaagtagacggcagcga

SEQ ID NO: 81 - DNA, 20 bp, Cucumis_sativus
tctttgggcattgaagtaga

SEQ ID NO: 82 - DNA, 20 bp, Cucumis_sativus
tcttgcagcagtttctcttt

SEQ ID NO: 83 - DNA, 20 bp, Cucumis_sativus
ttcttgcagcagtttctctt

SEQ ID NO: 84 - DNA, 20 bp, Cucumis_sativus
aaagagaaactgctgcaaga

SEQ ID NO: 85 - DNA, 20 bp, Cucumis_sativus
aaggcgttaaaaccgtcgtc

SEQ ID NO: 86 - DNA, 20 bp, Cucumis_sativus
cggtgaatccaccctcttca

SEQ ID NO: 87 - DNA, 20 bp, Cucumis_sativus
ggtgaatccaccctcttcaa

SEQ ID NO: 88 - DNA, 20 bp, Cucumis_sativus
ggccaagtgacccttattta

SEQ ID NO: 89 - DNA, 1385 bp, Cucumis sativus
acagtgtgtgagagtgtgctttatttcaaatctgaaagaaaaagaaagaaaaaaggattgaaattatggcaattagatcaaaagtaagatcaggtgagctgcagaatcctcttgttcttggaagagtaattggagatgttgttgatcccttcagtccaaccattaaaatgtctgtcactttcaccaataacaaacaagtcctcaatggccatgaattcttcccttcttctctttccttcaaacctagggttcatattcaaggagaagatatgagatcattgttcactctggtaaatcattatctttttttacctttttttttcttaaataaaagattgatagctttttgttttttttttttggttaggttatggttgaccctgatgttcctggccctagtgatccttacttgagggaacaccttcactggtatatatataaaaaacttccattcatttttaaattttgagctctctattgttgaaaaaaaaatttgaactttgagaaaacccatttccaaaatagtaatttctttctatttagagcctattttgtatgattttctcaatggtatttttttaaatatatatatatatatatatatatatatatatatatctttttttctattttaaagatgcaaaattgaaaacccattaccaaaacaataaatttatgtttgaatccaatttcaattttatcaacaaattggaaatggtaatgacctaactttttacctttatttggatctttttatttttcaggttggtgactgacattccaggaactactgatgctacttttggtaaggattactattttttaaaataaataaataaatgtatttgtgttttgaattaattttaattgaaaatgtttgggatgtggaatatatatatgagacaggaaaagaagaaatgagctatgaaattccaaagccaacaataggaattcacaggtttgtgtttattctgttcaaacaaaaacagcgtcggtcagtagtgaatcctccttcatcaagggatcgtttcaacactcgaagattttcttgtgagaatgatttgggtcttcctgttgctgctgtctatttcaatgctcaaagagaaactgctgcaaggaggcgataaatatcaaaatcatcatcatcttcgtcatcttcgtcatcttttaattttaatgccacatccatttatattttatcttcttctccattttaaattttaaatatatatatatatcaaaattcacaatctttatctatgtttatatatgtatcatgcattctaattttatggatgttttgaaaggataaatatttgttatccgctgttttttttccttatgttctatggatgcatttaatacaatttatttgatatagttat

SEQ ID NO: 90 - DNA, 549 bp, Cucumis sativus
atggcaattagatcaaaagtaagatcaggtgagctgcagaatcctcttgttcttggaagagtaattggagatgttgttgatcccttcagtccaaccattaaaatgtctgtcactttcaccaataacaaacaagtcctcaatggccatgaattcttcccttcttctctttccttcaaacctagggttcatattcaaggagaagatatgagatcattgttcactctggttatggttgaccctgatgttcctggccctagtgatccttacttgagggaacaccttcactggttggtgactgacattccaggaactactgatgctacttttggaaaagaagaaatgagctatgaaattccaaagccaacaataggaattcacaggtttgtgtttattctgttcaaacaaaaacagcgtcggtcagtagtgaatcctccttcatcaagggatcgtttcaacactcgaagattttcttgtgagaatgatttgggtcttcctgttgctgctgtctatttcaatgctcaaagagaaactgctgcaaggaggcgataa

SEQ ID NO: 91 - protein, 182 aa, Cucumis sativus
MAIRSKVRSGELQNPLVLGRVIGDVVDPFSPTIKMSVTFTNNKQVLNGHEFFPSSLSFKPRVHIQGEDMRSLFTLVMVDPDVPGPSDPYLREHLHWLVTDIPGTTDATFGKEEMSYEIPKPTIGIHRFVFILFKQKQRRSVVNPPSSRDRFNTRRFSCENDLGLPVAAVYFNAQRETAARRR

SEQ ID NO: 92 - DNA, 20 bp, Cucumis_sativus
gaaagaaaaagaaagaaaaa

SEQ ID NO: 93 - DNA, 20 bp, Cucumis_sativus
agaaaaaaggattgaaatta

SEQ ID NO: 94 - DNA, 20 bp, Cucumis_sativus
attagatcaaaagtaagatc

SEQ ID NO: 95 - DNA, 20 bp, Cucumis_sativus
attactcttccaagaacaag

SEQ ID NO: 96 - DNA, 20 bp, Cucumis_sativus
ctgcagaatcctcttgttct

SEQ ID NO: 97 - DNA, 20 bp, Cucumis_sativus
cttgttcttggaagagtaat

SEQ ID NO: 98 - DNA, 20 bp, Cucumis_sativus
attttaatggttggactgaa

SEQ ID NO: 99 - DNA, 20 bp, Cucumis_sativus
cattttaatggttggactga

SEQ ID NO: 100 - DNA, 20 bp, Cucumis_sativus
gtgacagacattttaatggt

SEQ ID NO: 101 - DNA, 20 bp, Cucumis_sativus
gaaagtgacagacattttaa

SEQ ID NO: 102 - DNA, 20 bp, Cucumis_sativus
attgaggacttgtttgttat

SEQ ID NO: 103 - DNA, 20 bp, Cucumis_sativus
aataacaaacaagtcctcaa

SEQ ID NO: 104 - DNA, 20 bp, Cucumis_sativus
ggaagaattcatggccattg

SEQ ID NO: 105 - DNA, 20 bp, Cucumis_sativus
gagaagaagggaagaattca

SEQ ID NO: 106 - DNA, 20 bp, Cucumis_sativus
gtttgaaggaaagagaagaa

SEQ ID NO: 107 - DNA, 20 bp, Cucumis_sativus
ggtttgaaggaaagagaaga

SEQ ID NO: 108 - DNA, 20 bp, Cucumis_sativus
aatatgaaccctaggtttga

SEQ ID NO: 109 - DNA, 20 bp, Cucumis_sativus
cttctctttccttcaaacct

SEQ ID NO: 110 - DNA, 20 bp, Cucumis_sativus
ttctctttccttcaaaccta

SEQ ID NO: 111 - DNA, 20 bp, Cucumis_sativus
tctccttgaatatgaaccct

SEQ ID NO: 112 - DNA, 20 bp, Cucumis_sativus
aaacctagggttcatattca

SEQ ID NO: 113 - DNA, 20 bp, Cucumis_sativus
tatgagatcattgttcactc

SEQ ID NO: 114 - DNA, 20 bp, Cucumis_sativus
cttttatttaagaaaaaaaa

SEQ ID NO: 115 - DNA, 20 bp, Cucumis_sativus
agctttttgttttttttttt

SEQ ID NO: 116 - DNA, 20 bp, Cucumis_sativus
ttttttttttggttaggtta

SEQ ID NO: 117 - DNA, 20 bp, Cucumis_sativus
cactagggccaggaacatca

SEQ ID NO: 118 - DNA, 20 bp, Cucumis_sativus
tcactagggccaggaacatc

SEQ ID NO: 119 - DNA, 20 bp, Cucumis_sativus
atggttgaccctgatgttcc

SEQ ID NO: 120 - DNA, 20 bp, Cucumis_sativus
aagtaaggatcactagggcc

SEQ ID NO: 121 - DNA, 20 bp, Cucumis_sativus
ccctcaagtaaggatcacta

SEQ ID NO: 122 - DNA, 20 bp, Cucumis_sativus
tccctcaagtaaggatcact

SEQ ID NO: 123 - DNA, 20 bp, Cucumis_sativus
tgaaggtgttccctcaagta

SEQ ID NO: 124 - DNA, 20 bp, Cucumis_sativus
ccctagtgatccttacttga

SEQ ID NO: 125 - DNA, 20 bp, Cucumis_sativus
acttgagggaacaccttcac

SEQ ID NO: 126 - DNA, 20 bp, Cucumis_sativus
ttttatatatataccagtga

SEQ ID NO: 127 - DNA, 20 bp, Cucumis_sativus
gctcaaaatttaaaaatgaa

SEQ ID NO: 128 - DNA, 20 bp, Cucumis_sativus
gaaattactattttggaaat

SEQ ID NO: 129 - DNA, 20 bp, Cucumis_sativus
agaaattactattttggaaa

SEQ ID NO: 130 - DNA, 20 bp, Cucumis_sativus
atagaaagaaattactattt

SEQ ID NO: 131 - DNA, 20 bp, Cucumis_sativus
ttgagaaaatcatacaaaat

SEQ ID NO: 132 - DNA, 20 bp, Cucumis_sativus
attttgtatgattttctcaa

SEQ ID NO: 133 - DNA, 20 bp, Cucumis_sativus
aaatttattgttttggtaat

SEQ ID NO: 134 - DNA, 20 bp, Cucumis_sativus
taaatttattgttttggtaa

SEQ ID NO: 135 - DNA, 20 bp, Cucumis_sativus
caaacataaatttattgttt

SEQ ID NO: 136 - DNA, 20 bp, Cucumis_sativus
tttgttgataaaattgaaat

SEQ ID NO: 137 - DNA, 20 bp, Cucumis_sativus
tttcaattttatcaacaaat

SEQ ID NO: 138 - DNA, 20 bp, Cucumis_sativus
ttttatcaacaaattggaaa

SEQ ID NO: 139 - DNA, 20 bp, Cucumis_sativus
ccaaataaaggtaaaaagtt

SEQ ID NO: 140 - DNA, 20 bp, Cucumis_sativus
cctaactttttacctttatt

SEQ ID NO: 141 - DNA, 20 bp, Cucumis_sativus
aaataaaaagatccaaataa

SEQ ID NO: 142 - DNA, 20 bp, Cucumis_sativus
tttggatctttttatttttc

SEQ ID NO: 143 - DNA, 20 bp, Cucumis_sativus
gatctttttatttttcaggt

SEQ ID NO: 144 - DNA, 20 bp, Cucumis_sativus
aggttggtgactgacattcc

SEQ ID NO: 145 - DNA, 20 bp, Cucumis_sativus
aaagtagcatcagtagttcc

SEQ ID NO: 146 - DNA, 20 bp, Cucumis_sativus
ggaactactgatgctacttt

SEQ ID NO: 147 - DNA, 20 bp, Cucumis_sativus
tactgatgctacttttggta

SEQ ID NO: 148 - DNA, 20 bp, Cucumis_sativus
taattttaattgaaaatgtt

SEQ ID NO: 149 - DNA, 20 bp, Cucumis_sativus
aattttaattgaaaatgttt

SEQ ID NO: 150 - DNA, 20 bp, Cucumis_sativus
aattgaaaatgtttgggatg

SEQ ID NO: 151 - DNA, 20 bp, Cucumis_sativus
gtggaatatatatatgagac

SEQ ID NO: 152 - DNA, 20 bp, Cucumis_sativus
tgaattcctattgttggctt

SEQ ID NO: 153 - DNA, 20 bp, Cucumis_sativus
gaaattccaaagccaacaat

SEQ ID NO: 154 - DNA, 20 bp, Cucumis_sativus
aacctgtgaattcctattgt

SEQ ID NO: 155 - DNA, 20 bp, Cucumis_sativus
agccaacaataggaattcac

SEQ ID NO: 156 - DNA, 20 bp, Cucumis_sativus
tgttcaaacaaaaacagcgt

SEQ ID NO: 157 - DNA, 20 bp, Cucumis_sativus
aaacgatcccttgatgaagg

SEQ ID NO: 158 - DNA, 20 bp, Cucumis_sativus
ttgaaacgatcccttgatga

SEQ ID NO: 159 - DNA, 20 bp, Cucumis_sativus
agtgaatcctccttcatcaa

SEQ ID NO: 160 - DNA, 20 bp, Cucumis_sativus
attttcttgtgagaatgatt

SEQ ID NO: 161 - DNA, 20 bp, Cucumis_sativus
ttttcttgtgagaatgattt

SEQ ID NO: 162 - DNA, 20 bp, Cucumis_sativus
ttgaaatagacagcagcaac

SEQ ID NO: 163 - DNA, 20 bp, Cucumis_sativus
ctcaaagagaaactgctgca

SEQ ID NO: 164 - DNA, 20 bp, Cucumis_sativus
aaagagaaactgctgcaagg

SEQ ID NO: 165 - DNA, 20 bp, Cucumis_sativus
tagtgaatcctccttcatca

SEQ ID NO: 166 - DNA, 20 bp, Cucumis_sativus
gccctagtgatccttacttg

SEQ ID NO: 167 - DNA, 1629 bp, Cucumis sativus
cccaagttaactcactctctctcaccagccctaaactacccttttgaaatactatcttgaaccttttttttatttcttttagtttctatggcgatggggaagatgccgtccgatcctctcgtggttggaggagtagtcggagatgttgtcgatgcaatttctcctacggttaagatgaccgtcacttaccattcaaacaagaaggtgtgcaatgggcatgagttgcttccaaattttgtaaccctaaaacctaaggttgaggttcttggaggtgaccttagatcattcttcacactggtctgatcatatactttcaatcaaacccatcttttcttttcttttctttttctaatgtatatataattagtattgatcaccgtacttaatttatatagtttcaactaattatttttttccaggtcatgactgatccagatgttcctggtccaagtgatccttacttgagagaacacctccactggtatcatattctaattcaagaaccccatctgtaagggtgtaaaaatgaatcttaattttcaaataaataaataagtttctataacaattgaactatgttaaagattgtcgaaacaagaatatgtcaagtattgtttgggaattggttgaatagtttttgttagaatattttgaacatgttacttagtgttctaaaaatcaaaatacaagggttttaggtttcaaaagtactttatatacgaacactaaaattttaaagggcatcttttgcaacttgtgtaatatatatctatctaatttttttctccaataaagaatgatttctctttatccttcgacttaactaacacatgttattagtaattaaatcatgtaaacttacgttttctctcgctttttgtaagtcctcgatttcaattgtataactactgaaataatttggataatatataactttgatatttcaggatagttacagacattccagggacgacggatgccacttttggtaaaacttcatttttttataattttcttctgataaattttgtttataatatatacatactagatactgatgggaaaattaataaaatataggaaaagagatagtaaaatatgaagaaccaagtccaaacatagggatacacagatatgtgttcctattgtacaagcaaaaaagaagacaaacagtgaagccgccaccacacccttcaagagacggttttaattcaagaaaatttgctcttgataatcatctttctcttcctgttgctgctgtctatttcattgctcaaaggcctactgctgccagaagaagataacacacacacacacatgtatatattcaacccagaattatatattctaaataaattattatattatatttcatcacataaattaaataaaattttgggttatatatatatatgaattttcatgtgtagcagagagaggcagtgaccttttcttgtgctgtcctttattgtgttctctttggtttatttgatgtactaaaactttgtatttctacttctctatgtatctgtttgtttaagttaattgtcttgtttttagttttctattgtttataaaaaaaaatcattttctttcccagaaagataatcaataaatgctttagtacat

SEQ ID NO: 168 - DNA, 537 bp, Cucumis sativus
atggcgatggggaagatgccgtccgatcctctcgtggttggaggagtagtcggagatgttgtcgatgcaatttctcctacggttaagatgaccgtcacttaccattcaaacaagaaggtgtgcaatgggcatgagttgcttccaaattttgtaaccctaaaacctaaggttgaggttcttggaggtgaccttagatcattcttcacactggtcatgactgatccagatgttcctggtccaagtgatccttacttgagagaacacctccactggatagttacagacattccagggacgacggatgccacttttggaaaagagatagtaaaatatgaagaaccaagtccaaacatagggatacacagatatgtgttcctattgtacaagcaaaaaagaagacaaacagtgaagccgccaccacacccttcaagagacggttttaattcaagaaaatttgctcttgataatcatctttctcttcctgttgctgctgtctatttcattgctcaaaggcctactgctgccagaagaagataa

SEQ ID NO: 169 - protein, 178 aa, Cucumis sativus
MAMGKMPSDPLVVGGVVGDVVDAISPTVKMTVTYHSNKKVCNGHELLPNFVTLKPKVEVLGGDLRSFFTLVMTDPDVPGPSDPYLREHLHWIVTDIPGTTDATFGKEIVKYEEPSPNIGIHRYVFLLYKQKRRQTVKPPPHPSRDGFNSRKFALDNHLSLPVAAVYFIAQRPTAARRR

SEQ ID NO: 170 - DNA, 20 bp, Cucumis_sativus
ttcaagatagtatttcaaaa

SEQ ID NO: 171 - DNA, 20 bp, Cucumis_sativus
gttcaagatagtatttcaaa

SEQ ID NO: 172 - DNA, 20 bp, Cucumis_sativus
aactaaaagaaataaaaaaa

SEQ ID NO: 173 - DNA, 20 bp, Cucumis_sativus
tttatttcttttagtttcta

SEQ ID NO: 174 - DNA, 20 bp, Cucumis_sativus
tcttttagtttctatggcga

SEQ ID NO: 175 - DNA, 20 bp, Cucumis_sativus
cttttagtttctatggcgat

SEQ ID NO: 176 - DNA, 20 bp, Cucumis_sativus
ttttagtttctatggcgatg

SEQ ID NO: 177 - DNA, 20 bp, Cucumis_sativus
ccaaccacgagaggatcgga

SEQ ID NO: 178 - DNA, 20 bp, Cucumis_sativus
tcctccaaccacgagaggat

SEQ ID NO: 179 - DNA, 20 bp, Cucumis_sativus
gatgccgtccgatcctctcg

SEQ ID NO: 180 - DNA, 20 bp, Cucumis_sativus
actactcctccaaccacgag

SEQ ID NO: 181 - DNA, 20 bp, Cucumis_sativus
ccgtccgatcctctcgtggt

SEQ ID NO: 182 - DNA, 20 bp, Cucumis_sativus
tccgatcctctcgtggttgg

SEQ ID NO: 183 - DNA, 20 bp, Cucumis_sativus
ctcgtggttggaggagtagt

SEQ ID NO: 184 - DNA, 20 bp, Cucumis_sativus
tgtcgatgcaatttctccta

SEQ ID NO: 185 - DNA, 20 bp, Cucumis_sativus
gtgacggtcatcttaaccgt

SEQ ID NO: 186 - DNA, 20 bp, Cucumis_sativus
cttgtttgaatggtaagtga

SEQ ID NO: 187 - DNA, 20 bp, Cucumis_sativus
tgcacaccttcttgtttgaa

SEQ ID NO: 188 - DNA, 20 bp, Cucumis_sativus
cacttaccattcaaacaaga

SEQ ID NO: 189 - DNA, 20 bp, Cucumis_sativus
tcaaacaagaaggtgtgcaa

SEQ ID NO: 190 - DNA, 20 bp, Cucumis_sativus
caaacaagaaggtgtgcaat

SEQ ID NO: 191 - DNA, 20 bp, Cucumis_sativus
ggttttagggttacaaaatt

SEQ ID NO: 192 - DNA, 20 bp, Cucumis_sativus
aacctcaaccttaggtttta

SEQ ID NO: 193 - DNA, 20 bp, Cucumis_sativus
gaacctcaaccttaggtttt

SEQ ID NO: 194 - DNA, 20 bp, Cucumis_sativus
ttttgtaaccctaaaaccta

SEQ ID NO: 195 - DNA, 20 bp, Cucumis_sativus
cctccaagaacctcaacctt

SEQ ID NO: 196 - DNA, 20 bp, Cucumis_sativus
aaccctaaaacctaaggttg

SEQ ID NO: 197 - DNA, 20 bp, Cucumis_sativus
aaacctaaggttgaggttct

SEQ ID NO: 198 - DNA, 20 bp, Cucumis_sativus
cctaaggttgaggttcttgg

SEQ ID NO: 199 - DNA, 20 bp, Cucumis_sativus
ccagtgtgaagaatgatcta

SEQ ID NO: 200 - DNA, 20 bp, Cucumis_sativus
ccttagatcattcttcacac

SEQ ID NO: 201 - DNA, 20 bp, Cucumis_sativus
aagaaaagaaaagaaaagat

SEQ ID NO: 202 - DNA, 20 bp, Cucumis_sativus
aaagaaaagaaaagaaaaga

SEQ ID NO: 203 - DNA, 20 bp, Cucumis_sativus
gaaactatataaattaagta

SEQ ID NO: 204 - DNA, 20 bp, Cucumis_sativus
tcaactaattatttttttcc

SEQ ID NO: 205 - DNA, 20 bp, Cucumis_sativus
catctggatcagtcatgacc

SEQ ID NO: 206 - DNA, 20 bp, Cucumis_sativus
tcacttggaccaggaacatc

SEQ ID NO: 207 - DNA, 20 bp, Cucumis_sativus
atgactgatccagatgttcc

SEQ ID NO: 208 - DNA, 20 bp, Cucumis_sativus
aagtaaggatcacttggacc

SEQ ID NO: 209 - DNA, 20 bp, Cucumis_sativus
tctctcaagtaaggatcact

SEQ ID NO: 210 - DNA, 20 bp, Cucumis_sativus
tggaggtgttctctcaagta

SEQ ID NO: 211 - DNA, 20 bp, Cucumis_sativus
acttgagagaacacctccac

SEQ ID NO: 212 - DNA, 20 bp, Cucumis_sativus
ttagaatatgataccagtgg

SEQ ID NO: 213 - DNA, 20 bp, Cucumis_sativus
gaattagaatatgataccag

SEQ ID NO: 214 - DNA, 20 bp, Cucumis_sativus
tttttacacccttacagatg

SEQ ID NO: 215 - DNA, 20 bp, Cucumis_sativus
atttttacacccttacagat

SEQ ID NO: 216 - DNA, 20 bp, Cucumis_sativus
catttttacacccttacaga

SEQ ID NO: 217 - DNA, 20 bp, Cucumis_sativus
ttcaagaaccccatctgtaa

SEQ ID NO: 218 - DNA, 20 bp, Cucumis_sativus
agaatatgtcaagtattgtt

SEQ ID NO: 219 - DNA, 20 bp, Cucumis_sativus
gaatatgtcaagtattgttt

SEQ ID NO: 220 - DNA, 20 bp, Cucumis_sativus
gtcaagtattgtttgggaat

SEQ ID NO: 221 - DNA, 20 bp, Cucumis_sativus
gttctaaaaatcaaaataca

SEQ ID NO: 222 - DNA, 20 bp, Cucumis_sativus
ttctaaaaatcaaaatacaa

SEQ ID NO: 223 - DNA, 20 bp, Cucumis_sativus
aatcaaaatacaagggtttt

SEQ ID NO: 224 - DNA, 20 bp, Cucumis_sativus
tacgaacactaaaattttaa

SEQ ID NO: 225 - DNA, 20 bp, Cucumis_sativus
acgaacactaaaattttaaa

SEQ ID NO: 226 - DNA, 20 bp, Cucumis_sativus
aaagagaaatcattctttat

SEQ ID NO: 227 - DNA, 20 bp, Cucumis_sativus
acatgtgttagttaagtcga

SEQ ID NO: 228 - DNA, 20 bp, Cucumis_sativus
tagttatacaattgaaatcg

SEQ ID NO: 229 - DNA, 20 bp, Cucumis_sativus
gtataactactgaaataatt

SEQ ID NO: 230 - DNA, 20 bp, Cucumis_sativus
atatataactttgatatttc

SEQ ID NO: 231 - DNA, 20 bp, Cucumis_sativus
aggatagttacagacattcc

SEQ ID NO: 232 - DNA, 20 bp, Cucumis_sativus
ggatagttacagacattcca

SEQ ID NO: 233 - DNA, 20 bp, Cucumis_sativus
aaagtggcatccgtcgtccc

SEQ ID NO: 234 - DNA, 20 bp, Cucumis_sativus
tacagacattccagggacga

SEQ ID NO: 235 - DNA, 20 bp, Cucumis_sativus
gggacgacggatgccacttt

SEQ ID NO: 236 - DNA, 20 bp, Cucumis_sativus
aaatgaagttttaccaaaag

SEQ ID NO: 237 - DNA, 20 bp, Cucumis_sativus
atatacatactagatactga

SEQ ID NO: 238 - DNA, 20 bp, Cucumis_sativus
tatacatactagatactgat

SEQ ID NO: 239 - DNA, 20 bp, Cucumis_sativus
tgggaaaattaataaaatat

SEQ ID NO: 240 - DNA, 20 bp, Cucumis_sativus
tgtatccctatgtttggact

SEQ ID NO: 241 - DNA, 20 bp, Cucumis_sativus
gaagaaccaagtccaaacat

SEQ ID NO: 242 - DNA, 20 bp, Cucumis_sativus
tatctgtgtatccctatgtt

SEQ ID NO: 243 - DNA, 20 bp, Cucumis_sativus
ttcttttttgcttgtacaat

SEQ ID NO: 244 - DNA, 20 bp, Cucumis_sativus
tctcttgaagggtgtggtgg

SEQ ID NO: 245 - DNA, 20 bp, Cucumis_sativus
ccgtctcttgaagggtgtgg

SEQ ID NO: 246 - DNA, 20 bp, Cucumis_sativus
aaaccgtctcttgaagggtg

SEQ ID NO: 247 - DNA, 20 bp, Cucumis_sativus
aattaaaaccgtctcttgaa

SEQ ID NO: 248 - DNA, 20 bp, Cucumis_sativus
gaattaaaaccgtctcttga

SEQ ID NO: 249 - DNA, 20 bp, Cucumis_sativus
ccaccacacccttcaagaga

SEQ ID NO: 250 - DNA, 20 bp, Cucumis_sativus
atgaaatagacagcagcaac

SEQ ID NO: 251 - DNA, 20 bp, Cucumis_sativus
ctgtctatttcattgctcaa

SEQ ID NO: 252 - DNA, 20 bp, Cucumis_sativus
tatcttcttctggcagcagt

SEQ ID NO: 253 - DNA, 20 bp, Cucumis_sativus
gtgtgtgtgttatcttcttc

SEQ ID NO: 254 - DNA, 20 bp, Cucumis_sativus
aagaaccaagtccaaacata

SEQ ID NO: 255 - DNA, 20 bp, Cucumis_sativus
attcaagaaccccatctgta